(19) United States
(12) Patent Application Publication
(10) Pub. No.: US 2023/0000396 A1
(43) Pub. Date: Jan. 5, 2023

(54) SYSTEMS AND METHODS FOR DETECTING MOVEMENT

(71) Applicants: ResMed Sensor Technologies Limited, Dublin 4 (IE); ResMed Pty Ltd, Bella Vista, NSW (AU); ResMed Corp., San Diego, CA (US)

(72) Inventors: Sam COFFEY, Dublin (IE); Michael WREN, Dublin (IE); Redmond SHOULDICE, Dublin (IE); Niall O'MAHONY, Dublin (IE); Andrew John PARTINGTON, Sydney (AU); Jacqueline Harrison SMITH, Sydney (AU); Charles Andrew William HARTSON, Sydney (AU); Rowan Ben FURLONG, Sydney (AU); Jose Ricardo DOS SANTOS, San Diego, CA (US); Jessica Elizabeth MONK, Sydney (AU)

(21) Appl. No.: 17/642,583

(22) PCT Filed: Sep. 11, 2020
(86) PCT No.: PCT/US20/50526

§ 371 Date: Mar. 11, 2022

Related U.S. Application Data
(60) Provisional application No. 62/900,277, filed on Sep. 13, 2019.
(60) Provisional application No. 62/902,374, filed on Sep. 18, 2019.
(60) Provisional application No. 62/955,934, filed on Dec. 31, 2019.
(60) Provisional application No. 63/023,361, filed on May 12, 2020.

(51) Int. Cl. A61B 5/11; A61B 5/00

(52) U.S. Cl. CPC A61B 5/1117; A61B 5/002; A61B 5/7267; A61B 5/7275

(57) ABSTRACT

A system includes a sensor configured to generate data associated with movements of a resident for a period of time, a memory storing machine-readable instructions, and a control system arranged to provide control signals to one or more electronic devices. The control system also includes one or more processors configured to execute the machine-readable instructions to analyze the generated data associated with the movement of the resident, determine, based at least in part on the analysis, a likelihood for a fall event to occur for the resident within a predetermined amount of time, and responsive to the determination of the likelihood for the fall event satisfying a threshold, cause an operation of the one or more electronic devices to be modified.

### CROSS-REFERENCE TO RELATED APPLICATIONS

This application claims the benefit of, and priority to, U.S. Provisional Patent Application No. 62/900,277 filed on Sep. 13, 2019 and entitled “MOVEMENT DETECTION,” U.S. Provisional Patent Application No. 62/902,374 filed on Sep. 18, 2019 and entitled “MOVEMENT DETECTION,” U.S. Provisional Application Patent No. 62/955,934 filed on Dec. 31, 2019 and entitled “SYSTEMS AND METHODS FOR DETECTING MOVEMENT,” and U.S. Provisional Patent Application No. 63/023,361 filed on May 12, 2020 and entitled “SYSTEMS AND METHODS FOR DETECTING MOVEMENT,” the disclosures of which are hereby incorporated by reference herein in their entirety.

### TECHNICAL FIELD

The present disclosure relates generally to systems and methods for predicting and preventing impending falls, and more particularly, to systems and methods for predicting and preventing impending falls of a resident of a facility (e.g., a hospital, an assisted living facility, or a home) using a sensor.

### BACKGROUND

Due to the aging population, falls are a major public health issue. Falls are a significant contributor to injury-related death in older adults. Moreover, falls can lead to chronic pain, disability, loss of independence, and high financial burden. Falls can occur during walking, from sitting to standing, or even when lying on an elevated surface (e.g., a bed). The present disclosure is directed to solving this and other problems.

### SUMMARY

According to some implementations of the present disclosure, a system includes a sensor, a memory, and a control system. The sensor is configured to generate data associated with movements of a resident for a period of time. The memory stores machine-readable instructions. The control system is arranged to provide control signals to one or more electronic devices and includes one or more processors configured to execute the machine-readable instructions to (i) analyze the generated data associated with the movement of the resident, (ii) determine, based at least in part on the analysis, a likelihood for a fall event to occur for the resident within a predetermined amount of time, and (iii) responsive to the determination of the likelihood for the fall event satisfying a threshold, cause an operation of the one or more electronic devices to be modified.

According to some implementations of the present disclosure, a system for predicting when a resident of a facility will fall includes a sensor, a memory, and a control system. The sensor is configured to generate current data and historical data associated with movements of a resident. The memory stores machine-readable instructions. The control system includes one or more processors configured to execute the machine-readable instructions to (i) receive as an input to a machine learning fall prediction algorithm the current data and (ii) determine as an output of the machine learning fall prediction algorithm a predicted time period in the future within which the resident is estimated to fall with a likelihood that exceeds a predetermined value.

According to some implementations of the present disclosure, a system for training a machine learning fall prediction algorithm includes a sensor, a memory, and a control system. The sensor is configured to generate data associated with movements or activity of a resident of a facility. The memory stores machine-readable instructions. The control system includes one or more processors configured to execute the machine-readable instructions to (i) accumulate the data, the data including historical data and current data, and (ii) train a machine learning algorithm with the historical data such that the machine learning algorithm is configured to (a) receive as an input the current data and (b) determine as an output a predicted time period or a predicted location at which the resident will experience a fall.

According to some implementations of the present disclosure, a method for predicting a fall using machine learning includes accumulating data associated with movements or activity of a resident of a facility. The data includes historical data and current data. A machine learning algorithm is trained with the historical data such that the machine learning algorithm is configured to (i) receive as an input the current data and (ii) determine as an output a predicted time period or a predicted location at which the resident will experience a fall.

According to some implementations of the present disclosure, a method for predicting when a resident of a facility will fall includes generating, via a sensor, current data and historical data associated with movements of a resident. The method further includes receiving as an input to a machine learning fall prediction algorithm the current data and determining as an output of the machine learning fall prediction algorithm a predicted time period in the future within which the resident is estimated to fall with a likelihood that exceeds a predetermined value.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system for generating physiological data associated with a user, according to some implementations of the present disclosure;

FIG. 2 is a perspective view of an environment, a resident walking in the environment, and a sensor monitoring the resident, according to some implementations of the present disclosure;

FIG. 3 is a perspective view of the environment of FIG. 2, where the resident is tripping with the sensor continuing to monitor the resident, according to some implementations of the present disclosure;

FIG. 4 is a perspective view of the environment of FIG. 2, where the resident has fallen to the ground as a result of the tripping shown in FIG. 3 with the sensor continuing to monitor the resident, according to some implementations of the present disclosure;

FIG. 5 is a perspective view of an environment, a resident lying in a configurable bed apparatus, and a sensor monitoring the resident, according to some implementations of the present disclosure;

FIG. 6 is a perspective view of the environment of FIG. 5, where the resident has rolled over to a first side of the configurable bed apparatus and the sensor continues to monitor the resident, according to some implementations of the present disclosure;

FIG. 7 is a perspective view of the environment of FIG. 5, where the configurable bed apparatus is adjusted to aid in preventing the resident from falling from the first side of the configurable bed apparatus, according to some implementations of the present disclosure;

FIG. 8 is a cross-sectional view of a footwear garment including one or more air bladders configured to inflate and/or deflate according to one or more schemes to aid in adjusting a gait of a resident wearing the footwear garment, according to some implementations of the present disclosure;

FIG. 9 is a cross-sectional view of the footwear garment of FIG. 8, where the one or more air bladders are at least partially inflated relative to FIG. 8, according to some implementations of the present disclosure;

FIG. 10 is a process flow diagram of a method for predicting when a resident of a facility will fall, according to some implementations of the present disclosure;

FIG. 11 is a process flow diagram of a method for training a machine learning fall prediction algorithm, according to some implementations of the present disclosure;

FIG. 12 is a schematic diagram depicting a computing environment, according to certain implementations of the present disclosure; and

FIG. 13 is a flowchart depicting a process for determining a falling inference, according to certain implementations of the present disclosure.

While the present disclosure is susceptible to various modifications and alternative forms, specific implementations thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many elderly people are at risk from a variety of hazards, such as falling, tripping, or illness. For example, health statistics and studies show that falling is a major problem among the elderly. The risk of falling increases with age, such that, studies suggest that about 32% of individuals above 65 years of age and 51% of individuals above 85 years of age fall at least once a year. In addition, many elderly people live alone. Therefore, the elderly are at additional risk that they may not be able to call for help or receive assistance in a timely manner after experiencing a fall or illness.

As a result, systems that enable a resident of a home to call for assistance from anywhere in a home have been developed. In systems such as the Personal Emergency Response Systems (PERS), the elderly or disabled individual wears a watch, pendent, or other like device and presses a button in the event of an emergency (e.g., a fall). The depressed button enables an alarm signal to be automatically sent to a central monitoring facility, when the resident has fallen. A disadvantage of using these devices is that they have to be worn by the person in order to work and are useless if the person is not wearing them or cannot activate them properly. Furthermore, these devices provide means to get help only after a fall has occurred. Thus, there is a risk that in an emergency situation, the resident may not receive proper assistance in a timely manner.

Certain systems rely on motion sensors to try to identify when a person has fallen. There may be extended periods where a resident is not moving for reasons other than the person having fallen or becoming incapacitated, such as watching television from a chair or sleeping in bed. Systems that rely on motion sensors require the person to be motionless for a considerable amount of time before the system is able to conclude that the resident has fallen or become incapacitated, as opposed to exhibiting normal inactive behavior.

Fall prevention screening techniques have also been used to identify a person's likelihood of falling. These techniques are traditionally performed through manual tests given by a trained professional, who determines the likelihood of fall risk for a person by identifying a set of typical fall risk factors that affect the person. A fall risk screening form is generally presented to the person that lists a set of possible fall risk factors for the person, and serves as a mechanism for the person to have these risk factors assessed by his/her therapist. A disadvantage with using fall risk screening techniques is that they are performed using manual tests that are only conducted periodically, such as for example, on a monthly basis. In addition, these techniques cannot be used to accurately predict future falls.

The present disclosure teaches systems and methods for predicting and preventing impending falls of a user in a facility using one or more sensors and in some implementations, one or more communicatively coupled devices. As used herein, the term facility is inclusive of various types of locations where a user may be living, whether permanently or temporarily, such as hospitals, assisted living communities, houses, apartments, and any other suitable location. The term facility is inclusive of care facilities (e.g., hospitals and assisted living communities) intended for providing ongoing, professional monitoring and/or treatment to a user, as well as a user's home (e.g., a house, apartment, and the like) in which, for example, a home health agency provides ongoing, professional monitoring and/or treatment to the user. The term facility is also inclusive of non-care facilities (e.g., houses, apartments, and the like) not intended for ongoing, professional monitoring and/or treatment of the user. A facility can be a single location (e.g., a single hospital or house) or can be a logical grouping of multiple locations (e.g., multiple hospitals or multiple houses). For example, a caregiver at a home health agency may provide services to multiple residents each located in their own house or apartment, in which case the caregiver may be able to monitor fall risk information for each of the residents on a centralized dashboard, despite each resident being located in a different house. The disclosed systems and methods allow for frequent monitoring of data and factors that increase the likelihood of falling of a resident, in real-time or substantially real-time. In addition, the disclosed systems and methods allow for automatically predicting the likelihood of a fall for a resident. By resident, it is meant to include any human person, regardless of duration of stay in a particular location. The resident can be a patient in a hospital or any other care facility. Further, the resident can be a human living at home in a house, an apartment, a retirement community, a skilled nursing facility, an independent living facility, etc.

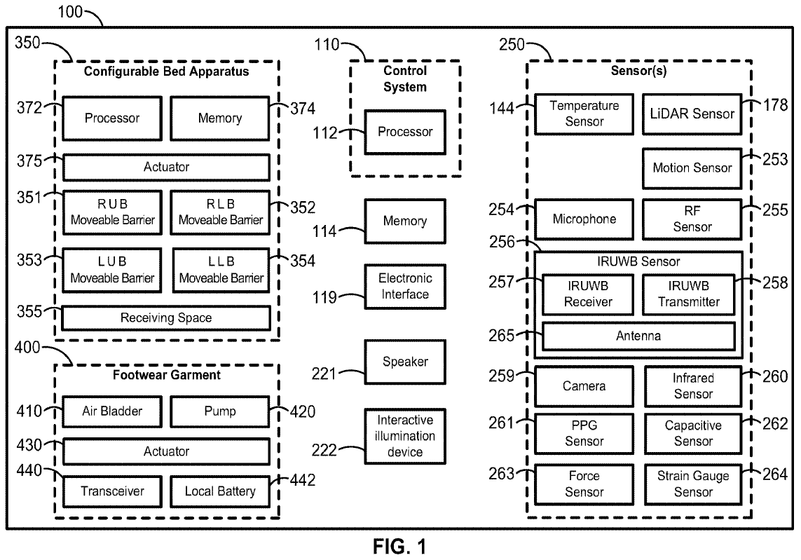

Referring to FIG. 1, a system 100 includes a control system 110, a memory device 114, a configurable bed apparatus 350 (which may include sensors as disclosed herein), a footwear garment 400, and one or more sensors 250. As described herein, the system 100 generally can be used to frequently monitor data and factors that can be indicative of an increase in a likelihood of a resident falling. The system 100 can also be used to predict when a resident is likely to fall (e.g., a likelihood of fall for a resident). The system 100 generally can also be used to aid in preventing the falling of a resident (e.g., in real-time). While the system 100 is shown as include various elements, the system 100 can include any subset of the elements shown and described herein and/or the system 100 can include one or more additional elements not specifically shown in FIG. 1. For example, in some cases, the configurable bed apparatus 350 and/or the footwear garment 400 are optionally not included, and other elements can be optionally included.

The control system 110 includes one or more processors 112 (hereinafter, processor 112). The processors 112 can be operatively coupled to a memory device 114. In some cases, the memory device 114 is separate from the control system 100, however that need not always be the case. The control system 110 is generally used to control (e.g., actuate) the various components of the system 100 and/or analyze data obtained and/or generated by the components of the system 100. The processor 112 executes machine readable instructions that are stored in the memory device 114 and can be a general or special purpose processor or microprocessor. While one processor 112 is shown in FIG. 1, the control system 110 can include any suitable number of processors (e.g., one processor, two processors, five processors, ten processors, etc.). The memory device 114 can be any suitable computer readable storage device or media, such as, for example, a random or serial access memory device, a hard drive, a solid state drive, a flash memory device, etc. While one memory device 114 is depicted in FIG. 1, any number of memory devices 114 can be used. The control system 110 can be coupled to and/or positioned within a housing of the one or more sensor 250, the configurable bed apparatus 350, the footwear garment 400, a speaker 221, an interactive illumination device 222, or any combination thereof. The control system 110 can be centralized (within one housing) or decentralized (within two or more physically distinct housings). In some cases, the control system 110 can be implemented across multiple computing devices (e.g., smart sensors and/or computers), although that need not always be the case.

The configurable bed apparatus 350 includes a processor 372, a memory 374, an actuator 375, a right upper body moveable barrier 351, a right lower body moveable barrier 352, a left upper body moveable barrier 353, a left lower body moveable barrier 354, and a receiving space 355. It should be understood that the barriers 351, 352, 353, and 354 can be configured to move in various ways and/or can be configured to have various other shapes and sizes, etc. For example, the configurable bed apparatus 350 can include a single left moveable barrier and a single right moveable barrier, or three or more barriers on each side. Furthermore, the configurable bed apparatus 350 can include additional features (e.g., movable mattress portion(s), one or more movable pillows, etc.).

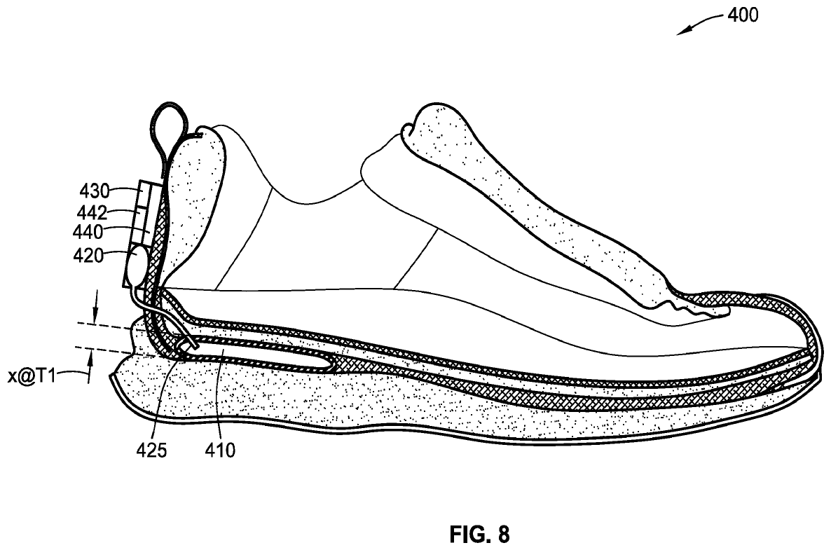

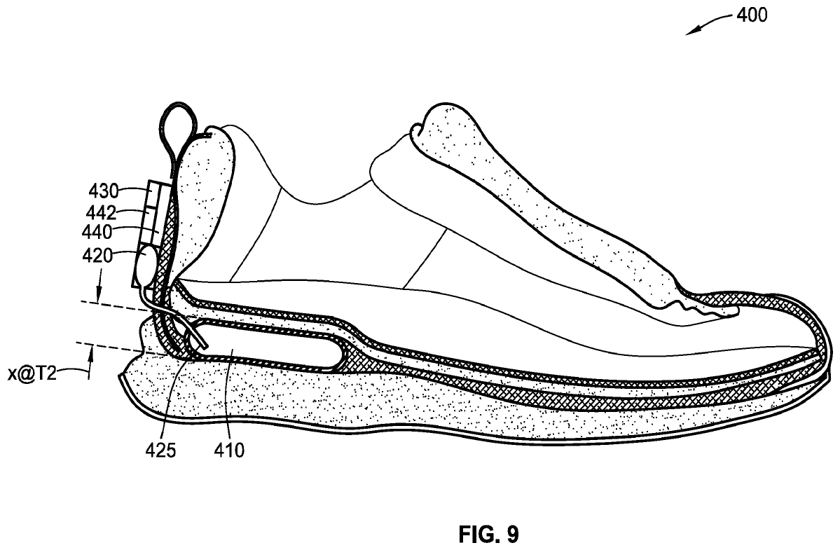

The footwear garment 400 includes an air bladder 410 coupled to a pump 420 by a tube 425 (FIGS. 8 and 9). The footwear garment 400 can also include an actuator 430, a transceiver 440, and a local battery 442. It should be understood that the footwear garment 400 can include a single sneaker, or a pair of sneakers. The disclosed implementation can be incorporated in other types of footwear, such as, for example, boots, slippers, loafers, casual, business, and orthopedic shoes. Furthermore, the footwear garment 400 can include additional features. The transceiver 440 of the footwear garment 400 is communicatively coupled (e.g., wireless communication) to the control system 110.

The one or more sensors 250 include a temperature sensor 252, a motion sensor 253, a microphone 254, a radio-frequency (RF) sensor 255, an impulse radar ultra wide band (IRUWB) sensor 256, a camera 259, an infrared sensor 260, a photoplethysmogram (PPG) sensor 261, a capacitive sensor 262, a force sensor 263, a strain gauge sensor 264, a Light Detection and Ranging (LiDAR) sensor 178, or any combination thereof. Generally, each of the one or more sensors 250 are configured to output sensor data that is received and stored in the memory device 114 of the control system 110. An RF sensor could be an FMCW (Frequency Modulated Continuous Wave) based system or system on chip where the frequency increases linearly with time (e.g., a chirp) with different shapes such as triangle (e.g., frequency swept up, then down), sawtooth (e.g., frequency ramp swept up or down, then reset), stepped or non-linear shape and so forth. The sensor may use multiple chirps that do not overlap in time or frequency, with one or more transmitters and receivers. It could operate at or around any suitable frequencies, such as at or around 24 GHz, or at or around millimeter wave (e.g., 76-81 GHz) or similar frequencies. The sensor can measure range as well as angle and velocity.

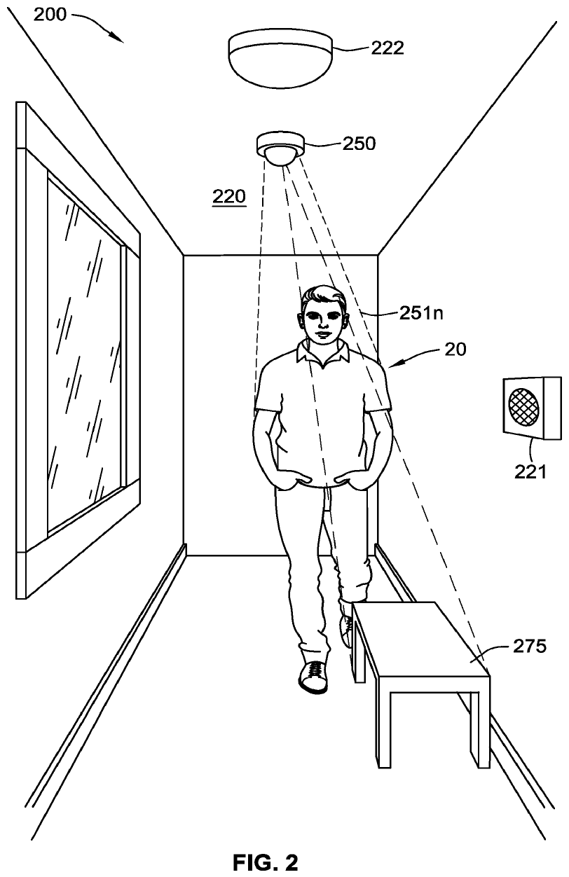

The IRUWB sensor 256 includes an IRUWB receiver 257 and an IRUWB transmitter 258. The IRUWB transmitter 258 generates and/or emits radio waves having a predetermined frequency and/or a predetermined amplitude (e.g., within a high frequency band, within a low frequency band, long wave signals, short wave signals, etc., or any combination thereof). The IRUWB receiver 257 detects the reflections of the radio waves emitted from the IRUWB transmitter 258, and the data can be analyzed by the control system 110 to determine a location of a resident (e.g., resident 20 of FIG. 2) and a state of the resident (e.g., standing, sitting, falling, fallen, running, walking, lying down on an object, lying down on the floor/ground, etc.). The IRUWB receiver 257 and/or the IRUWB transmitter 258 can be wirelessly connected with the control system 110, one or more other devices (e.g., the configurable bed apparatus 350, the footwear garment, etc.), or both. While the IRUWB sensor 256 is shown as having a separate IRUWB receiver and IRUWB transmitter in FIG. 1, in some implementations, the IRUWB sensor 256 can include a transceiver that acts as both the IRUWB receiver 257 and the IRUWB transmitter 258.

Specifically, the IRUWB sensor 256 is configured to transmit, receive and measure the timing between short (e.g., nominally nanosecond) impulses of radio waves. Thus, the IRUWB sensor 256 is short-range in nature and is highly affected by objects in the propagation path. The sensor data can be analyzed by one or more processors 112 of the control system 110 to calibrate the one or more sensors 250, to frequently monitor data and factors that increase the likelihood of a resident falling, to train a machine learning algorithm, or any combination thereof.

In some implementations of the present disclosure, the IRUWB sensor 256 is and/or includes an Impulse Radio Ultra Wide Band (IR-UWB or IRUWB) RADAR that emits electromagnetic radio waves (e.g., occupying >500 MHz and/or 25% of the fractional bandwidth) and receives the reflected waves from one or more objects. Using such a sensor, it is possible to detect movements of one or more objects. The detected one or more objects can include long term stationary objects (e.g., static objects like a bed, a dresser, a wall, a ceiling, etc.), as well as objects that move occasionally, that move frequently, or that move periodically. Using the IRUWB sensor 256 it is possible to track moving objects with a high degree of precision within the environment in which the object is moving. The wide bandwidth of the signal along with very short duration impulses allows for high resolution sensing and multipath capability, along with RF co-existence.

It should be understood that the one or more sensors 250 can include any combination and any number of the sensors described and/or shown herein. The temperature sensor 252 outputs temperature data that can be stored in the memory device 114 of the control system 110 and/or analyzed by the processor 112 of the control system 110. In some implementations, the temperature sensor 252 generates temperatures data indicative of a core body temperature of a resident (e.g., resident 20 of FIG. 2), a skin temperature of the resident, an ambient temperature, or any combination thereof. The temperature sensor 252 can be, for example, a thermocouple sensor, a thermistor sensor, a silicon band gap temperature sensor or semiconductor-based sensor, a resistance temperature detector, or any combination thereof.

The motion sensor 253 can detect motion of one or more objects in a space (e.g., a resident, such as resident 20 of FIG. 2). In some implementations, the motion sensor 253 is a Wi-Fi base station or a high frequency 5G mobile phone that includes controller software therein to sense motion. For example, a Wi-Fi node in a mesh network can be used for motion sensing, using subtle changes in RSS (receive signal strength) across multiple channels. Further, such motion sensors 253 can be used to process motion from multiple targets, breathing, heart, gait, fall, behavior analysis, etc. across an entire home and/or building and/or hospital setting.

The microphone 254 outputs sound data that can be stored in the memory device 114 of the control system 110 and/or analyzed by the processor 112 of the control system 110. The microphone 254 can be used to record sound(s) related to falls and/or gait/walking of a resident (e.g., resident 20 of FIG. 2) to determine, for example, information about the type of fall, a degree of severity of the fall, whether certain sounds were heard after the fall (e.g., movement sounds, cries for help, sounds of inbound assistance), stride parameters, etc. Examples of different types of fall include cataclysmic, moderate fall, braced fall, stumble, trip and recover, trip and fall, etc.

The speaker 221 outputs sound waves that are audible to the resident (e.g., resident 20 of FIG. 2). The speaker 221 can be used, for example, as an alarm clock and/or to play an alert or message to the resident (e.g., in response to a fall event) and/or to a third party (e.g., a family member of the resident, a friend of the resident, a caregiver of the resident, etc.). In some implementations, the microphone 254 and the speaker 221 can be used collectively used together as a sonar sensor. In such implementations, the speaker 221 generates or emits sound waves at a predetermined interval and the microphone 254 detects the reflections of the emitted sound waves from the speaker 221. The sound waves generated or emitted by the speaker 221 have a frequency that is not audible to the human ear, which can include infrasound frequencies (e.g., at or below approximately 20 Hz) and/or ultrasonic frequencies (e.g., at or above approximately 18-20 kHz) so as not to disturb the resident (e.g., resident 20 of FIG. 2). Based at least in part on the data from the microphone 254 and the speaker 221, the control system 110 can determine a location of the resident, the state of the resident, one or more cough events, one or more physiological parameters, and/or one or more of the sleep-related parameters, as described in herein.

The RF sensor 255 includes one or more RF transmitters, one or more RF receivers, and a control circuit. The RF transmitter generates and/or emits radio waves having the predetermined frequency and/or a predetermined amplitude. The RF receiver detects the reflections of the radio waves emitted from the RF transmitter, and the data can be analyzed by the control system 110 to determine a location of a resident (e.g., resident 20 of FIG. 2). The RF sensor 255 can also be used to monitor physiological parameters, one or more cough events, and/or one or more of the sleep-related parameters of the resident, as described in herein. In some cases, the RF sensor 255 can be a frequency modulated continuous wave (FMCW) transceiver array. In some cases, several sensors in communication with each other and/or a central system (such as in the facility or in the cloud) may be used to cover the desired area to be monitored—such as bedroom, hall, bathroom, kitchen, sitting room, and the like.

The RF receiver and/or the RF transmitter can also be used for wireless communication between the control system 110, the interactive illumination device 222, the speaker 221, the configurable bed apparatus 350, the footwear garment 400, the one or more sensors 250, or any combination thereof.

Examples and details of the RF sensor 255 and/or related sensors are described in, for example, WO2015/006364, WO2016/170005, WO2017/032873, WO2018/050913, WO2010/036700, WO2010/091168, WO2008/057883, WO20071143535, and U.S. Pat. No. 8,562,526, each of which is hereby incorporated by reference herein in its entirety.

The camera 259 outputs image data reproducible as one or more images (e.g., still images, video images, thermal images, or a combination thereof) that can be stored in the memory device 114 of the control system 110. The image data from the camera 259 can be used by the control system 110 to determine a location and/or a state of a resident (e.g., resident 20 of FIG. 2).

The infrared (IR) sensor 260 outputs infrared image data reproducible as one or more infrared images (e.g., still images, video images, or both) that can be stored in the memory device 114 of the control system 110. The infrared data from the IR sensor 260 can be used to determine a location and/or state of a resident (e.g., resident 20 of FIG. 2). The IR sensor 260 can also be used in conjunction with the camera 259 when measuring movement of the resident. The IR sensor 260 can detect infrared light having a wavelength between about 700 nm and about 1 mm, for example, while the camera 259 can detect visible light having a wavelength between about 380 nm and about 740 nm.

One or more Light Detection and Ranging (LiDAR) sensors 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 178 can measure and map an area extending 5 meters or more away from the sensor. The LiDAR data can be fused with point cloud data estimated by an electromagnetic RADAR sensor, for example. The LiDAR sensor(s) 178 may also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

The PPG sensor 261 outputs physiological data associated with a resident (e.g., resident 20 of FIG. 2) that can be used to determine a state of the resident. The PPG sensor 261 can be worn by the resident and/or embedded in clothing and/or fabric that is worn by the resident. The physiological data generated by the PPG sensor 261 can be used alone and/or in combination with data from one or more of the other sensors 250 to determine the state of the resident.

The capacitive sensor 262, the force sensor 263, and the strain gauge sensor 264 output data that can be stored in the memory device 114 of the control system 110 and used by the control system 110 individually and/or in combination with data from one or more other sensors 250 to determine a state of a resident (e.g., resident 20 of FIG. 2). In some implementations, the one or more sensors 250 also include a galvanic skin response (GSR) sensor, an electrocardiogram (ECG) sensor, an electroencephalography (EEG) sensor, an electromyography (EMG) sensor, a blood flow sensor, a respiration sensor, a pulse sensor, a sphygmomanometer sensor, an oximetry sensor, an oxygen sensor, a mattress sensor such as a PVDF sensor (stretchable polyvinylidene fluoride sensor that may be in strips or a serpentine layout) or force sensitive resistors, textile sensors, or any combination thereof.

The electronic interface 119 is configured to receive data (e.g., physiological data) from the one or more sensors 250 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, a Personal Area Network, over a cellular network (such as 3G, 4G/LTE, 5G), etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. The electronic interface 119 can also communicatively couple to the configurable bed apparatus 350, footwear garment 400, and/or any other controllable device to pass signals (e.g., data) to and/or from the control system 110. In some implementations, the electronic interface 119 is coupled to or integrated (e.g., in a housing) with the control system 110 and/or the memory device 114, although that need not always be the case.

While shown separately in FIG. 1, the one or more sensors 250 can be integrated in and/or coupled to any of the components of the system 100 including the control system 110, the external devices (e.g., the configurable bed apparatus 350, the footwear garment 400), or any combination thereof. For example, the microphone 254 and the speaker 221 can be integrated in and/or coupled to the control system 110, the configurable bed apparatus 350, the footwear garment 400, or a combination thereof. In some cases, the configurable bed apparatus 350 can include one or more sensors, such as a piezoelectric sensor, a PVDF sensor, a pressure sensor, a force sensor, an RF sensor, a capacitive sensor, and any combination thereof. In some implementations, at least one of the one or more sensors 250 are not coupled to the control system 110, or the external devices, and are positioned generally adjacent to a resident (e.g., resident 20 of FIG. 2) (e.g., coupled to or positioned on a nightstand, coupled to a mattress, coupled to a ceiling, coupled to a wall, coupled to a lighting device, etc.).

The one or more processors 112 of the control system 110 (FIG. 1) are configured to execute the machine-readable instructions to analyze the generated data associated with the movement of the resident 20 (FIGS. 2-4). The processors 112 are also configured to determine, based at least in part on the analysis, a likelihood for a fall event to occur for the resident 20 within a predetermined amount of time. The one or more processors 112 are also configured to execute the machine-readable instructions to cause an operation of the one or more electronic devices to be modified in response to the determination of the likelihood for the fall event satisfying a threshold (e.g., a threshold of likeliness of a fall or a threshold of expected severity of a likely fall). The control system 110 can send a command to the speaker 221 (FIGS. 1-4) to provide auditory guidance to prevent the resident 20 from falling. Such auditory guidance can include a warning of the static object 275, a warning to slow down, a warning to brace for impact, a warning to sit and rest, a warning not to get out of bed too quickly (as the resident may otherwise faint), a warning that there may be a level of risk in going into a shower unaided based on the current or historical biometrics of the resident, a warning that the room configuration has recently changed (e.g. a chair may have been moved earlier in the day into the typical pathway taken by the resident to the bathroom during the night), or any combination thereof. Other guidance can be provided. The control system 110 can send a command to a multi-colored interactive illumination device, such as interactive illumination device 222 (FIGS. 1-4), to modify a color or an intensity of the illuminated light.

Referring to FIG. 2, an environment 200 is illustrated where a resident 20 is walking down a hallway. The environment 200 also includes a static object 275. The static object 275 can include a bench, a chair, a sofa, a table, a lighting fixture, a rug, or any object within the environment that a control system (e.g., control system 110 of FIG. 1) determines is not the resident or another person. As shown, a sensor 250 is configured to detect, via transmitted signals 251n, a position of the resident 20. Sensor 250 as depicted in FIGS. 2-4 is an example of a suitable sensor of the one or more sensors 250 of FIG. 1, although other types or combinations of sensors can be used. The environment 200 can be a resident's home (e.g., house, apartment, etc.), an assisted living facility, a hospital, etc. Other environments are contemplated. The sensor 250 is mounted to a ceiling surface 220 of the environment 200, although the sensor 250 can be mounted to any surface in the environment 200 (e.g., to a wall surface, to a door, to a floor, to a window, etc.) or otherwise positioned in the environment 200. In some cases, the sensor 250 can be incorporated into a device such as a television, an alarm clock, a fan housing, an electrical fixture, a piece of furniture (e.g., a bed frame), a mirror, a toilet cistern, a sink or sink cabinet, a smoke or heat detector, or the like. That is, it should be understood that the sensor 250 can be mounted on other surfaces or otherwise positioned in the environment 200 to be able to perceive the resident 20. For example, the sensor 250 can be mounted on a vertical surface (wall). In some cases, the sensor 250 may be within view of the resident 20, although that need not always be the case. For example, some sensors (e.g., RF sensors) can sense through different surfaces, and may cover multiple rooms from one sensor array in order to reduce wiring complexity. In some cases, a sensor or array of sensors may make use of direct path sensing or multipath sensing (e.g., sensing using differing time of flight for different frequencies). Depending on the frequencies used, RF sensors may be able to “see through” stud walls, but not necessarily walls made from masonry blocks. Surfaces such as glass (e.g., windows, mirrors) may appear as reflective surfaces, and sensor data can be pre-processed to account for such reflective surfaces. Curtains and other fabrics may be transparent or substantially transparent to RF sensors, although it may be beneficial to cancel out certain motion (e.g., movement of curtains). In some cases, the sensor(s) may be mobile or movable, in order to make it easier to retrofit into an existing house, such as without requiring drilling of walls or hardwiring electrical connections. Such sensors can be installed by the end user or a nurse for example. The sensor 250 can also be positioned on a lower surface, such as a table, or a counter top. It should be understood that the position of the sensor 250 in the environment 200 is not intended to be exclusive. The sensor 250 is configured to generate data (e.g., location data, position data, physiological data, etc.) that can be used by the control system (e.g., control system 110 of FIG. 1) to determine a status of the resident 20. As shown in FIG. 2, the control system is able to receive the data generated by the sensor 250 and determine that the resident 20 is walking down the hallway and approaching the static object 275.

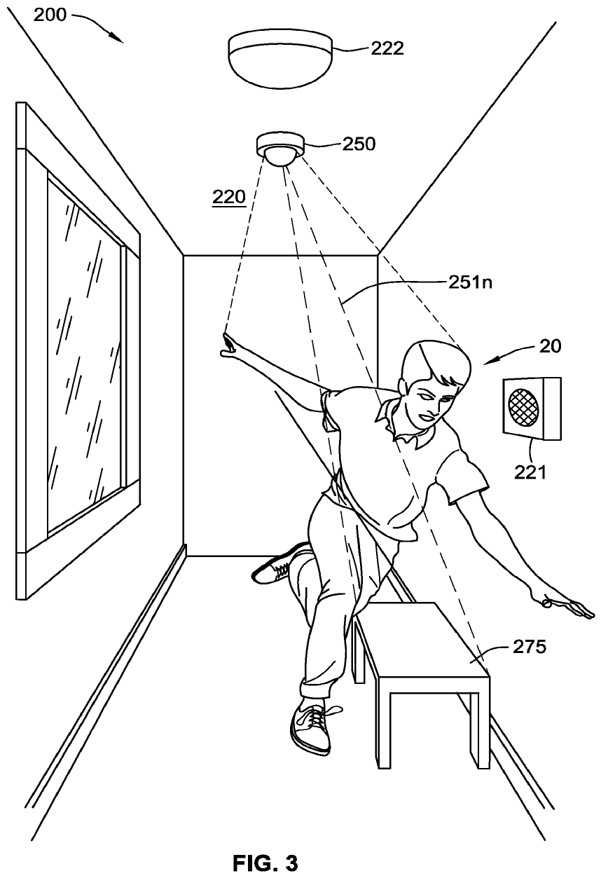

Referring to FIG. 3, the environment 200 of FIG. 2 is shown with the resident 20 in the process of falling or tripping. Specifically, FIG. 3 illustrates the sensor 250 generating data that can be processed by the control system (e.g., control system 110 of FIG. 1) to determine that the resident 20 in the process of falling (e.g., after bumping into the static object 275). That is, the sensor 250 is configured to generate data associated with movements and/or activities of the resident 20 for a period of time. Specifically, the sensor 250 is configured to detect the resident 20 and their state. For example, the sensor 250 is configured to detect whether the resident is standing, lying, sitting up, walking, etc. The sensor 250 is also able to observe the resident 20 over time to generate historical data. Some information the sensor 250 is able to detect and observe includes the time it takes for the resident 20 to get out of bed and/or walking from one room to another room.

Where more than one sensor (or more than one sensor array) is used, including the possibility for different sensor modalities covering different regions of a space, the sensors may communicate with each other to reduce or eliminate mutual interference where active sensing (such as RF signals, light signals, etc.) is used. This inter-sensor communication via wired or wireless means can allow the sensing frequency ranges to not overlap in time and space, for example between multiple devices.

Where more than one sensor (or more than one sensor array) is used, the possibility of using different sensor modalities to cover different regions of a space is possible, such as to make sure there are no important regions that lack coverage (e.g., a sensor over bed may not be able to reliably detect a fall behind a chair or sofa, but a further away sensor at a higher elevation and/or different frequency range may be able to directly sense the region behind the chair or sofa to detect a fall there).

In some versions of an RF sensor, a processor such as a microcontroller may be configured to select a subset of ranges by controlling the sensor to automatically scan through a superset of potential ranges by adjusting a range setting of the sensor. The selection of a range of the subset of ranges may be based on a detection of any one or more of bodily movement, respiration movement, and/or cardiac movement in the range of the subset of ranges. The microcontroller may be configured to control range selection to discretely implement a gesture-based user interface range and a physiological signal detection range. The microcontroller may be configured to control range gating to initiate a scan through a plurality of available ranges upon determination of an absence of any one or more of previously detected bodily movement, respiration movement and/or cardiac movement in a detection range. The microcontroller may be configured to control the range gating of the initiated scan through the plurality of available ranges of the range gating while detecting any one or more of bodily movement, respiration movement and/or cardiac movement in a different detection range of the range selection.

In some versions, dynamic range management, under control of the processor, may be implemented to follow the movement of one or more residents within a sensing space (e.g., an environment). For example, a user may roll over in a bed and potentially leave a sensing area defined by a current range of the range gating or selection area of the sensor. This change in position may be material to the fall risk assessment artificial intelligence model. Upon detection of a change or loss in the sensing of physiological characteristics in the sensor signal (e.g., a detection of a loss or absence of previously detected motion or physiological motion) of that range, the sensor may adjust or scan through different available ranges by adjusting the range gating to locate a range where such motion (e.g., body motion or physiological motion) is present/detected. Alternatively, a local or remote processor may process the full sensor data streams, and make range selection determination, biometrics estimation, and pathway estimation (e.g. if the resident is moving around the space and defining a pathway). If such sensing is not again detected in any of the available ranges (and if no other sensing is occurring in any available range), such as after a predetermined time interval, the microcontroller may control a power supply to depower the sensor circuits (e.g., sensor transceiver circuits) to reduce power consumption and/or reduce usage of data bandwidth. In some cases, the sensor may periodically repower the transceiver circuits and rescan through the ranges to select a detection range for sensing if such motion is detected in an available range.

Such dynamic range selection and optional range gating or geofencing may involve multiple residents. For example, in some cases, the sensor, under control of the microcontroller, may scan through available ranges when motion is no longer detected from a first resident in one range, while continuing to sense motion of a second resident (or second person such as a caregiver, nurse, etc.) in a different range. Thus, the sensor may change the range gating (e.g., scan through available ranges other than the range of the second resident) to detect motion of the first resident in another available range and continue sensing of the first resident upon detection of motion in such an available range, while maintaining sensing in the gated range being used for the second resident. These dynamic range gating adjustments may be made by interleaving or multiplexing detection ranges by making programmatic adjustments to RF characteristics such as chirp configuration, antenna selection, beam forming, pulse timing implemented with the microcontroller controlled range gating of the transceiver, and so forth. Detection of significant physiological motion in any of the particular ranges may then serve as a basis for monitoring the physiological characteristics in the particular range. For example, if a resident moved closer to the sensor and the detection of the significant physiological motion (e.g., cardiac frequency, respiratory frequency) at the closer range may initiate or continue the focus of monitoring at the closer range setting.

For example, timing of the transceiver may be controlled by the microcontroller for implementing a variable range gating operation with a plurality of detection ranges by changing a range gating setting to change the sensor to monitor a resident in a second range when the resident moves to the second range from a first range previously monitored by the sensor. Optionally, timing of the transceiver may be controlled by the microcontroller for implementing a variable range gating operation with a plurality of detection ranges to substantially monitor a resident upon a change in the resident's location within the ranges of the sensor.

Thus, the sensor may be configured, such as with the timing settings of the dynamic range gating, to monitor the physiological characteristics of any of, for example: (a) one or more stationary residents; (b) one or more residents, where at least one of them is changing their location; (c) one or more residents who have just entered the range of the sensor; (d) one or more residents where one is stationary or otherwise, and another resident who has just entered the range of the sensor and is stationary or otherwise, etc.

In some embodiments, it may be desirable to pre-configure the range settings. For example, if a sensor array is mounted at a bed, its range settings can be pre-set at the factory to a standard two-in bed king size bed with the sensor placed on a bedside locker or night stand in a mobile configuration. These settings may be configurable, e.g. by the resident, caregiver, or installer, etc., through a controller, such as through a software application (e.g., an app) on a smartphone. Additionally, these ranges can be automatically optimized by the system, using movement, activity, respiration, and heart (i.e. from ballistocardiogram) features. For example, a subset of ranges may be automatically determined/selected, such as in a setup or initial operation procedure, by controlling the sensor to automatically scan or iterate through a larger superset of potential ranges by changing the range settings (e.g., magnitude detector receive timing pulse(s)). The selection of the detection range subset (e.g., one or more) can be dependent on detection of any one or more of the body movement or other human activity, respiration (respiration movement) and/or heartbeat (cardiac movement) features in a particular range(s). The selected subset of ranges may then be used during a detection session (e.g., a night of sleep).

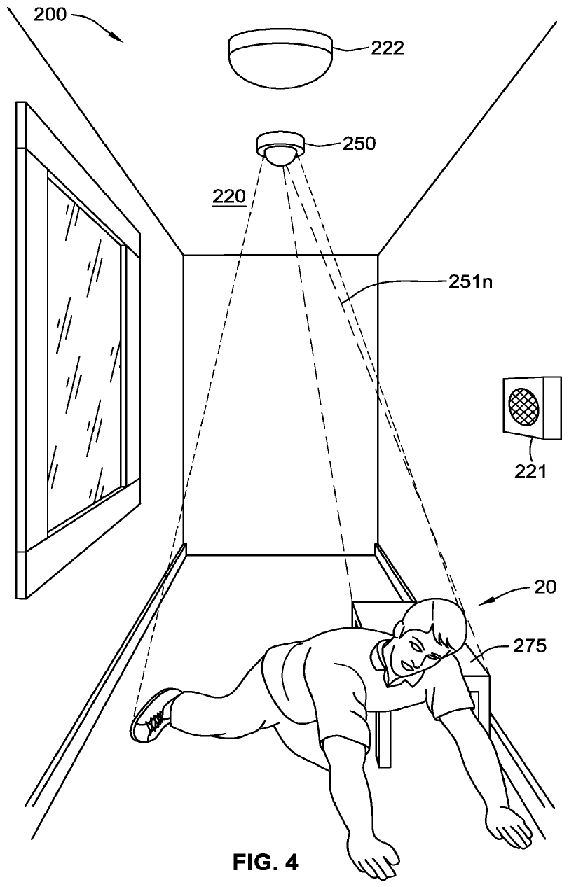

Referring to FIG. 4, the environment 200 of FIGS. 2 and 3 is shown where the resident 20 has fallen due to tripping on the static object 275. Similar to above, the sensor 250 generates data that can be analyzed by a control system 110 to determine that the resident 20 experienced a fall. The control system 110 can send a command to the speaker 221 to assure the resident 20 that help is on the way. The control system 110 can send a command to the speaker 221 to inquire about the health/injury of the resident 20. This fall can be documented by the system 100 as historical data associated with the resident 20 and be used in future analyses to predict future falls of the resident 20. In some cases, historical data of one resident 20 can be used to inform fall prediction for other residents as well, such as residents with similar characteristics (e.g., similar age, similar biological traits, similar conditions, similar walking patterns or movement patterns, and the like). The system can listen (e.g., via a microphone) for a response (e.g., from the resident 20) to the voice command using natural language processing or other voice recognition, and check if the alert is real or a false alarm. Other actions can be taken, as disclosed herein.

The sensor 250 is shown in the environment 200 of FIGS. 2-4 and described herein as generating data that can be processed and/or analyzed by a control system (e.g., control system 110 of FIG. 1). In some implementations, the control system is contained within and/or coupled to the same housing that contains the sensor 250 shown in FIGS. 2-4. Alternatively, the sensor 250 of FIGS. 2-4 is electronically connected (wirelessly and/or wired) to a separate control system, which is positioned elsewhere in the environment 200, in a cloud system, in a server system, in a computer, in the same facility as the sensor 250, etc., or any combination thereof.

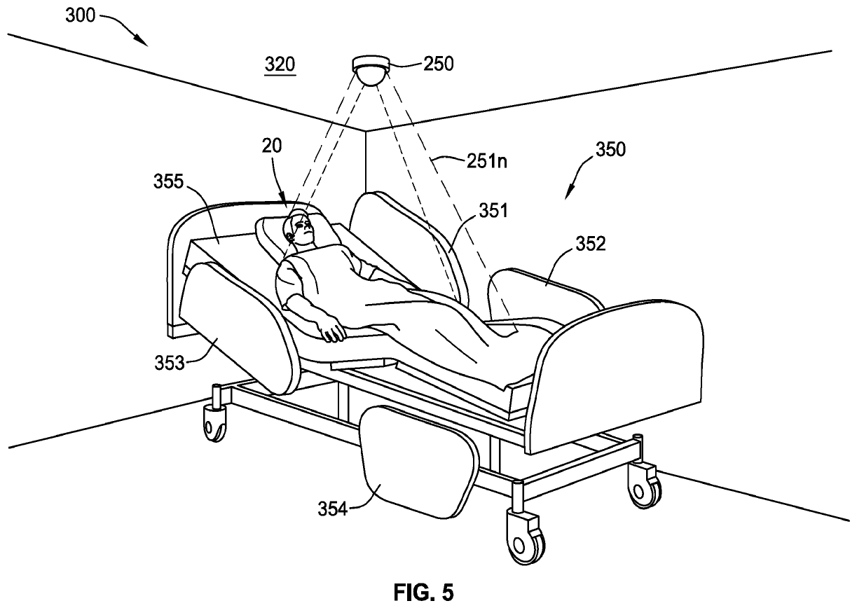

Referring to FIG. 5, an environment 300 where the resident 20 is located in the receiving space 355 of the configurable bed apparatus 350 is shown. A sensor 250 is configured to generate data using transmitted signals 251n. Sensor 250 as depicted in FIGS. 2-4 is an example of a suitable sensor of the one or more sensors 250 of FIG. 1, although other types or combinations of sensors can be used. The generated data can be analyzed and/or processed by a control system (e.g., control system 110 of FIG. 1) to determine a position of the resident 20 within the receiving space 355 of the configurable bed apparatus 350. The environment 300 can be a resident's home (e.g., house, apartment, etc.), an assisted living facility, a hospital, etc. Other environments are contemplated. The sensor 250 is mounted to a ceiling surface 320 of the environment 300, although the sensor 250 can be mounted to any surface in the environment 300 (e.g., to a wall surface, to a door, to a floor, to a window, etc.) or otherwise located in the environment. That is, it should be understood that the sensor 250 can be mounted on other surfaces or otherwise positioned to be able to perceive the resident 20. For example, the sensor 250 can be mounted on a vertical surface (wall). In some cases, the sensor 250 may be within view of the resident 20, although that need not always be the case. The sensor 250 can also be positioned on a lower surface, such as a table, or a counter top. It should be understood that the position of the sensor 250 in the environment 300 is not intended to be exclusive. The sensor 250 is configured to generate data (e.g., location data, position data, physiological data, etc.) that can be used by the control system to determine a status of the resident 20. As shown in FIG. 5, the control system is able to receive the data generated by the sensor 250 and determine that the resident 20 is generally centrally positioned within the receiving space 355 of the configurable bed apparatus 350 or otherwise positioned at a distance from an edge of the receiving space 355. However, if the resident 20 were to approach an edge of the receiving space 355, the sensor 250 is configured to generate data indicative of a change of position of the resident 20.

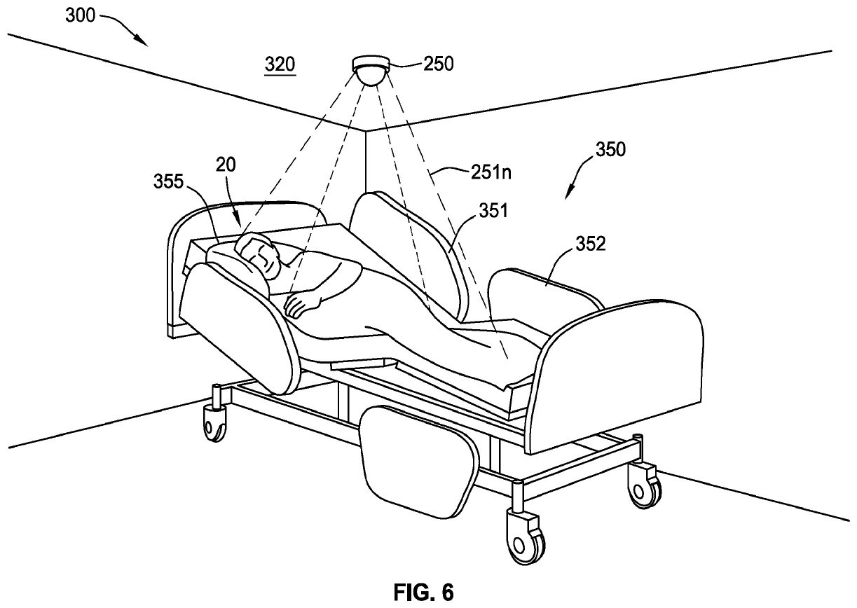

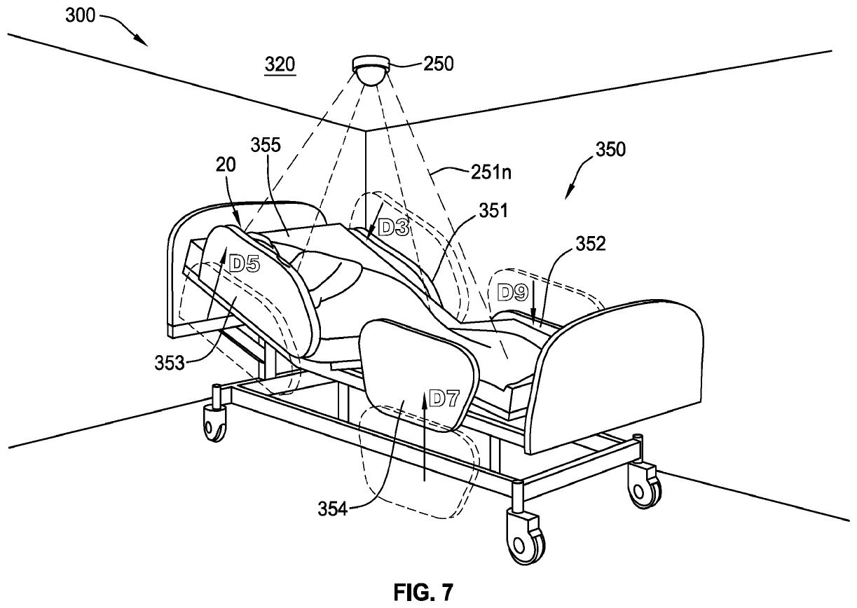

Referring to FIG. 6, the environment 300 of FIG. 5 is shown with the resident 20 rolled over towards a first edge of the configurable bed apparatus 350. The sensor 250 is configured to generate data associated with movements and activities of the resident 20 for a period of time. Referring to FIG. 7, based at least in part on the data generated by the sensor 250 that is indicative of the resident 20 being positioned adjacent to an edge of the configurable bed apparatus 350, the control system causes one or more control signals to be sent to the configurable bed apparatus 350. The one or more control signal sent to the configurable bed apparatus 350 cause the actuator 375 (shown in FIG. 1) of the configurable bed apparatus 350 to trigger (e.g., move) one or more moveable barriers. The moveable barriers are triggered in response to the determination (e.g., by the control system 110) that a likelihood for a fall event to occur (e.g., the resident 20 falling out of the configurable bed apparatus 350) satisfies a threshold.

In some implementations of the present disclosure, as shown in FIG. 7, the left upper body moveable barrier 353 and the left lower body moveable barrier 354 are actuated into a deployed or upward position in response to the control system determining that the threshold is satisfied, which indicates that a fall event is likely to occur imminently (e.g., the resident 20 is likely to fall off the left edge of the configurable bed apparatus 350 as viewed in FIG. 7). In the process of actuating the left upper body moveable barrier 353 and the left lower body moveable barrier 354, the opposite right upper body moveable barrier 351 and the right lower body moveable barrier 352 can also be retracted such that the resident 350 is not entrapped in the configurable bed apparatus 350.

While FIGS. 5-7 relate to the use of certain actuatable barriers (e.g., left upper body movable barrier 353, right upper body movable barrier 351, left lower body movable barrier 354, and right lower body movable barrier 352) to reduce a likelihood of or otherwise prevent an imminent fall based on the determination by the control system, other actuatable devices can be used in an environment (e.g., environment 300). Other actuatable devices can be used in conjunction with a bed, such as inflatable pillow or mattress regions, or in conjunction with other devices (e.g., walls, floor, chairs, doors, sofas, railings, etc.) in the environment 300 that may be able to affect the likelihood that the resident 20 will fall, such as to reduce a likelihood of or otherwise prevent an imminent fall based on the determination by the control system.

Referring to FIG. 8, a cross-sectional view of the footwear garment 400 is shown. Footwear garment 400 of FIG. 8 is an example of footwear garment 400 of FIG. 1, although any other suitable footwear garment can be used. Similar to implementations aforementioned, a control system (e.g., control system 110 of FIG. 1) can analyze data from one or more sensors (e.g., one or more sensors 250 of FIG. 1) and determine a gait for the resident (e.g., resident 20 of FIG. 2) wearing the footwear garment 400. The determined gait for the resident wearing the footwear garment 400 can be indicative of a future fall event if not corrected and/or addressed. In some implementations, the system (e.g., system 100 of FIG. 1) can address a concern with a gait or parameter of a gait of a resident by adjusting one or more aspects of the footwear garment 400. In some such implementations, the gait can be specifically addressed in response to a concern that the gait will lead to the resident having a future fall if not addressed or otherwise corrected. In such implementations, the system causes the pump 420 to actuate to fill one or more sub-compartments in the air bladder 410, thereby modifying the sole of the footwear garment 400 to directly impact the gait of the resident wearing the footwear garment 400. The control system can send the necessary signals/command to the transceiver 440. In response to the appropriate signals/command, the actuator 430 can activate the pump 420 to inflate the air bladder 410 via one or more of the tubes 425. While one compartment is shown generally in the heal location of the footwear garment 400, it is contemplated that any number of compartments can be included at any position within the footwear garment 400. For example, the air bladder 410 can include 1, 2, 3, 4, 5, 10, 15, 20, 50, 100, 1000, etc. sub-compartments. In such implementations, each sub-compartment can be individually addressable. In some cases, each sub-compartment can be individually supplied with fluid, or can be fluidly connected in series and/or parallel with one or more supplies (e.g., tubes 425) from the pump 420. Further, the air bladder 410 or any portion thereof can be positioned adjacent to a heal portion of the footwear garment 400, a toe portion of the footwear garment 400, one or both side portions of the footwear garment 400, a central portion of the footwear garment 400, etc., or any combination thereof.

In some cases, one or more of the pump 420, actuator 430, transceiver 440, and local battery 442 can be detachable from the footwear garment 400. In some cases, elements of the footwear garment 400 associated with affecting gait on demand (e.g., pump 420, actuator 430, transceiver 440, local battery 442, tube(s) 425, and air bladder 410) can be separately provided (e.g., in the form of a removable insole) for integration into a resident's shoes or for swapping between shoes. In some cases, the transceiver 440 of a left shoe can be paired to a transceiver 440 of a right shoe to facilitate affecting a resident's gait, although that need not always be the case.

Referring to FIG. 9, the footwear garment 400 is shown with the inflated air bladder 410 inflated (e.g., inflated more than in FIG. 8). The air bladder 410, now inflated, is intended to provide support and/or adjustment to the resident (e.g., resident 20 of FIG. 2) when walking. As can be seen by a comparison of FIG. 8 and FIG. 9, the air bladder 410 has a first level of inflation or height x@T1 and a second level of inflation or height at x@T2. In some implementations, the adjustment is made to aid in preventing the resident from falling while walking. In some implementations, the adjustment is made to aid in modifying the resident's gait to aid in strengthening one or more muscles of the resident. Such targeted strengthening of muscles can be tailored to reduce a future risk of falling or can be used other purposes, such as physical therapy. In some implementations, the adjustment is made to change the speed and/or direction of movement of a resident to reduce the likelihood of, or otherwise prevent, collision with an object, such static object 275, in the path of the resident, wherein the resident and object are detected by the one or more sensors as described herein.

For example, in some implementations, a control system (e.g., control system 110 of FIG. 1) can determine that the likelihood for a resident to fall exceeds a threshold, and can then cause the air bladder 410 to inflate according to an inflation scheme to cause a modification to the gait of the resident. This modification to the gait of the resident can be strategically provided in one or both of the shoes of the resident to aid the resident to continue to walk while lowering the likelihood for the fall event to occur. The inflation scheme can be static (e.g., providing a single change to the air bladder(s) intended to remain steady through numerous steps) or dynamic (e.g., providing dynamic adjustment to the air bladder(s) as the user ambulates). While a footwear garment is disclosed herein, it should be understood that other walking assistance devices can also be implemented. For example, a resident (e.g., the resident shown in FIG. 2) with health conditions that limit their physical mobility, can use physical assistance devices to aid movement. These devices include walking sticks, walkers, wheelchairs, canes, and other similar devices. Such physical assistance devices can be configured with one or more actuators configured to affect the gait of the resident in response to a signal from a control system that has determined a need for gait modification.

In some cases, such footwear garments 400 and physical assistance devices can be configured with one or more of the sensors (e.g., one or more sensors 250 of FIG. 1) to monitor one or more aspects of the resident (e.g., movement, gait, stride, standing time, sitting time, etc., or any other one or more metrics described herein). In some implementations, the one or more sensors generate data that can be processed by the control system to determine whether the resident has fallen and/or to predict that the user is about to fall with a certain amount of time (e.g., within a week, two weeks, etc.). The generated data can include detected vibrations and/or movement patterns of the device.

In some such implementations, a smart walking stick is provided for use by a resident. The smart walking stick can include one or more of the sensors 250 described herein in connection with FIG. 1. In some implementations, the smart walking stick can also include a control system (e.g., control system 110 of FIG. 1) and/or a portion of a control system.

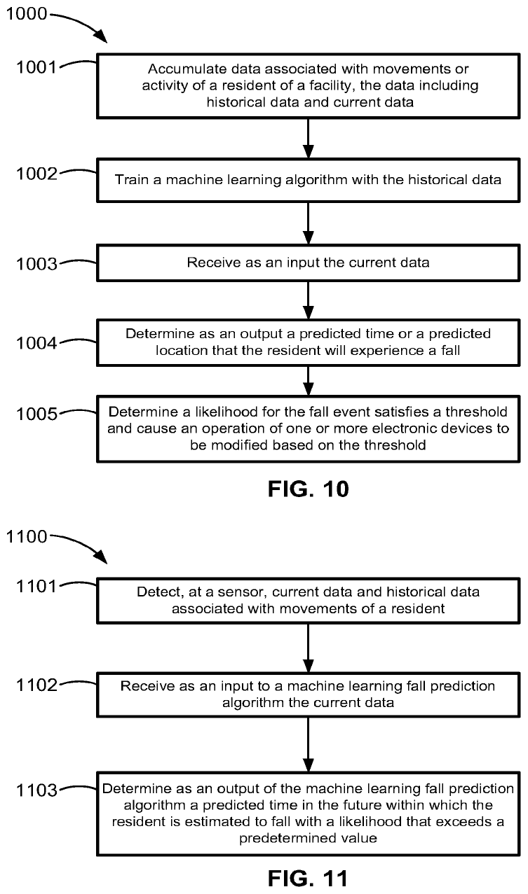

Referring to FIG. 10, a process flow diagram for a method 1000 of predicting when a resident of a facility will fall is shown. One or more of the steps of the method 1000 described herein can be implemented using the system 100 (FIG. 1). Step 1001 of the method 1000 includes accumulating data associated with movements or activity of a resident of a facility. The data can include historical data and current data. For example, step 1001 can include detecting movements and fall events for the resident, other people, static objects, or any combination thereof. The current and/or historical data can include the resident trying to get out of bed, walking from one room to another room, an amount of time it takes the resident to go from point A (a first point or location) to point B (a second point or location) in their environment, an amount of time it takes the resident to get out of bed, an amount of time it takes the resident to get out of a chair, an amount of time it takes the resident to get out of a couch, a shortening of a stride of the resident over time, a deterioration of a stride of the resident over time, etc. or any combination thereof.

Step 1002 of the method 1000 includes training a machine learning algorithm with the historical data. In such implementations, the current data can be received as input at step 1003. Based on that input, a predicted time and/or a predicted location that the resident will experience a fall is determined as an output. Step 1004 of the method 1000 includes determining the output.

This information can be used by the machine-learning algorithm over the course of multiple iterations of the method 1000 to aid in predicting when a resident of a facility will fall by, for example, receiving as current data the time it takes the resident to go from point A to point B in their environment (step 1003) and determine a predicted time and/or a predicted location that the resident will experience a fall (step 1004). If the machine-learning algorithm determines that certain current data is not affecting the difference in the fall analysis, these movements and/or objects are no longer observed in subsequent iterations of the method 1000. Thus, using the machine-learning algorithm can reduce the number of iterations of the method 1000 (prediction during step 1004) that are needed to predict when a resident of a facility will fall.

Step 1005 of the method 1000 includes determining if a likelihood of a fall event occurring satisfies a threshold and causing an operation of one or more electronic devices to be modified based on the threshold being satisfied. As discussed above with respect to FIGS. 1-9, the one or more electronic devices can include a speaker 221, an interactive illumination device 222, a configurable bed apparatus 350, a footwear garment 400, or any combination thereof.

Referring to FIG. 11, a process flow diagram for a method 1100 of training a machine learning fall prediction algorithm is shown. One or more of the steps of the method 1100 described herein can be implemented using the system 100 (FIG. 1) or any portion of the system 100. Step 1101 of the method 1100 includes generating, using a sensor (e.g., one or more of sensors 250), current data and historical data associated with movements of a resident. As discussed above, the sensor can include a temperature sensor 252, a motion sensor 253, a microphone 254, a radio-frequency (RF) sensor 255, an impulse radar ultra wide band (IRUWB) sensor 256, a camera 259, an infrared sensor 260, a photoplethysmogram (PPG) sensor 261, a capacitive sensor 262, a force sensor 263, a strain gauge sensor 264, or any combination thereof.

Step 1102 of the method 1100 includes receiving as an input to a machine learning fall prediction algorithm the current data. In some implementations, the current data can include movements and/or fall events for the resident, other people, static objects, or any combination thereof. The current data can also include the resident trying to get out of bed, walking from one room to another room, an amount of time it takes the resident to go from point A to point B in their environment, an amount of the time it takes the resident to get out of bed, an amount of time it takes the resident to get out of a chair, an amount of time it takes the resident to get out of a couch, a shortening of a stride of the resident over time, a deterioration of a stride of the resident over time, or any combination thereof.

Step 1103 of the method 1100 includes determining, as an output of the machine learning fall prediction algorithm, a predicted time in the future, where the resident is estimated to fall before such predicted time. Further, the occurrence of the fall before such predicted time has a likelihood of occurring that satisfies a threshold (e.g., exceeds a predetermined value). The output of the machine learning fall prediction algorithm can include an assessment, a rating, or any understanding of the risk of a fall for the resident. In some implementations of the present disclosure, the output can include a fall risk score that is assessed based on a defined threshold. Furthermore, in other implementations, the output can include a fall risk rating. In environments where there may exist multiple residents, the machine learning fall prediction algorithm can output a fall risk rating or likelihood of falling (e.g., within a predetermined amount of time) for each resident. In some cases, each resident can be detected by a unique biometric footprint of the resident. Such a biometric footprint can be any combination of biometric traits (e.g., a combination of height and breath rate) capable of being sensed by the system 100 and usable to uniquely identify an individual. Such information can be used by the system 100 to create a priority listing for therapy for the residents and/or to create a stoplight system for aiding in preventing one or more of the residents from falling. In one example, a 3-tier “stoplight” ranking system can denote each resident in the facility as a “high,” “medium,” or “low” risk resident in terms of the likelihood of incurring a fall.

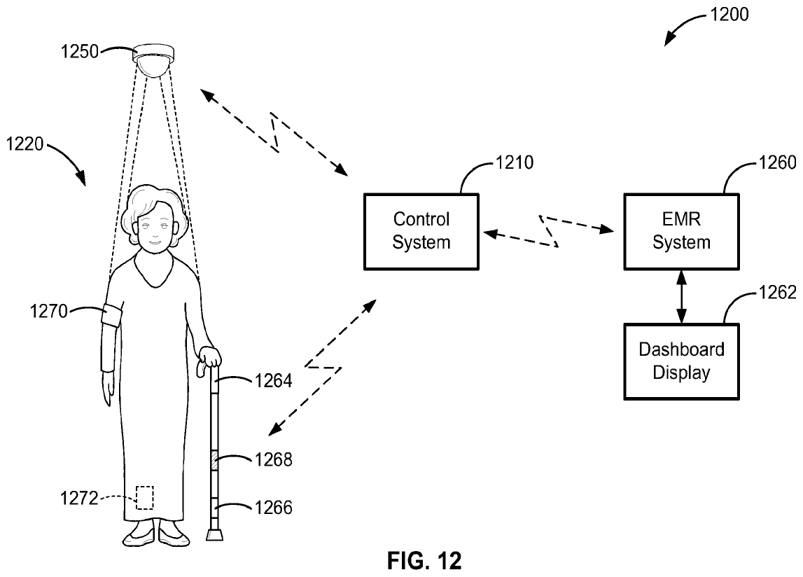

FIG. 12 is a schematic diagram depicting a computing environment 1200, according to some aspects of the present disclosure. The computing environment 1200 can include one or more sensors 1250 (e.g., one or more sensors 250 of FIG. 1) communicatively coupled to a control system 1210 (e.g., control system 110 of FIG. 1). The control system 1210 can receive signals (e.g., data) from the sensor(s) 1250, which can then be used to make a determination about the likelihood that a resident 1220 (e.g., resident 20 of FIG. 2-4 or 5-7) may fall (e.g., a fall inference).

As described with reference to FIGS. 8-9, in some cases, the control system 1210 can provide signal(s) to an assistance device 1264, such as a footwear garment, a smart walking stick, or other such physical assistance device. As depicted in FIG. 12, the physical assistance device in the form of a walking stick, although other forms can be used. The signal from the control system 1210, upon receipt by the assistance device 1264, can cause actuation of an actuatable element 1266 (e.g., an air pump, movable weight, or other suitable actuator) to affect the gait of the resident 1220. In some cases, the assistance device 1264 can include one or more sensors 1268 (e.g., one or more sensors 250 of FIG. 1) that are also communicatively coupled to the control system 1210 to provide further information about the resident 1220, such as position, use of the assistance device, gait information, and the like.

In some cases, the computing environment 1200 can include an electronic health record (EHR— such as a longitudinal collection of the electronic health information) such as an EMR (electronic medical record—patient record) system 1260 communicatively coupled to the control system 1210. The EHR may be connected to a personal health record (PHR) maintained by the patient themselves. The EMR may include Fast Healthcare Interoperability Resources (FHIR), derived from Health Level Seven International (HL7), to provide open, granular access to medical information. The EMR system 1260 can be implemented separate from the control system 1210, although that need not always be the case. For example, the EMR system 1260 can be implemented on a facility's intranet or can be implemented in a cloud or on an internet such as the Internet. In some cases, the EMR system 1260 can be communicatively coupled to a dashboard display 1262, which can be a display provided to practitioners and/or caregivers based on information in the EMR system 1260. For example, a dashboard display 1262 can include information about which residents are in the facility, where each resident is located in the facility, what medications each resident may be taking, any diagnoses associated with each resident, and any other such medical information, whether current or historical. While an EMR system 1260 is depicted and described with reference to FIG. 12, any other suitable computing system for storing, accessing, and/or displaying the resident's medical data can be used in place of the EMR system 1260.

The EMR system 1260 can communicate with the control system 1210 to share information related to the resident. In some cases, the control system 1210 can receive medical data about the resident from the EMR system 1260, which the control system 1210 can use in making its determination about the likelihood that a resident 1220 may fall. In an example, the EMR system 1260 can provide information that a particular resident is taking a medication that is likely to make the resident dizzy, in which case the control system 1210 can use this information to improve its determination that the resident 1220 is likely to fall. For example, when detecting that resident moving around the facility, the control system 1210 may have otherwise predicted that the resident 1220 is not likely to fall based on the resident's gait being sensed by the control system 1210, but because the control system 1210 now knows of the dizziness-inducing medication from the EMR system 1260, the control system 1210 may now determine that the resident 1220 exhibiting the sensed gait while on the dizziness-inducing medication has a high likelihood of falling. In some cases, the control system 1210 can transmit information to the EMR system 1260 for storage and/or further use by the EMR system 1260. In an example, the control system 1210 can send, to the EMR system 1260, information about an identified fall event or a determined likelihood for the resident to fall. The EMR system 1260 can store this information alongside the resident's medical information, such as to facilitate review by a practitioner or caregiver. In some cases, the EMR system 1260 can use the information from the control system 1210 to update the dashboard display 1262. For example, a dashboard display 1262 providing dashboard information associated with one or more residents in a facility or a portion of a facility can include both medical information from the EMR system 1260 and fall-related information (e.g., identification of a fall event, a determined likelihood of a fall occurring in the future, and/or a reason for why a likelihood of a fall occurring in the future has changed) from the control system 1210. In an example, the dashboard display 1262 can provide a tiered ranking system for the fall risk of the cohort, or portion thereof, of residents in a facility. In one example, a 3-tier “stoplight” ranking system can denote each resident in the facility as a “high,” “medium,” or “low” risk resident in terms of the likelihood of incurring a fall. Thus, a practitioner and/or caregiver reviewing the dashboard display can quickly identify which residents may need increased attention with respect to potential fall risks as compared to those residents with a low, or relatively lower, risk of falling. The practitioner and/or caregiver can then assign facility resources appropriately, without wasting valuable resources in preventing falling of a resident with an already low likelihood of falling.

In some cases, a wearable device 1270 can be communicatively coupled to the control system 1210. The wearable device 1270 can be any device capable of sensing and/or tracking biometric or health-related data of the resident. The control system 1210 can use sensor data from the wearable device 1270 to further inform its determination of a fall event or a likelihood of falling. In an example depicted in FIG. 12, the wearable device 1270 can be a wearable blood pressure monitor (e.g., automatic sphygmomanometer) capable of determining the blood pressure of the resident. In this example, the control system 1210 can use the blood pressure data from the wearable device 1270 in combination with the sensor data from sensor(s) 1250 to determine that the resident's blood pressure has dropped significantly (e.g., a drop in systolic blood pressure of, or greater than, 20 mmHg and/or a drop in diastolic blood pressure of, or greater than, 10 mmHg) after moving from a lying or seated position to a standing position. If the blood pressure drop (e.g., of systolic and/or diastolic) is over a threshold amount, the control system 1210 may make an inference that a fall event is likely to occur.

A second type of wearable device is also depicted in FIG. 12 as a leg strap 1272 that monitors the leg of the resident 1220. More specifically, the leg strap 1272 can monitor reflex motion and muscle tension, such as to infer leg strength, which can be used to generate a fall inference. The leg strap 1272 can be communicatively coupled to the control system 1210.

In practice, the one or more sensors 1250 can operate on a schedule or continuously to collect sensor data from an environment (e.g., a region of a room, a room, a set of rooms, a facility, a set of facilities, or other). Such sensor data can be indicative of persons (e.g., resident 1220) moving in and around the environment. Separately, practitioners and caregivers, whether manually or through automated tools, can provide updates to a EMR system 1260 in the form of updated health information (e.g., blood pressure monitoring, medication prescriptions and (re)fills, activity of daily life, and the like). The control system 1210 can access data from sensor 1250 to generate a fall inference (e.g., inference that a fall has occurred and/or that a fall is likely to occur), optionally using data from the EMR system 1260. An algorithm can be used to combine data from sensor 1250 and data from the EMR system 1260 to generate the fall inference. In an example, the algorithm can take into account at walking speed, sway, pathway deviations, heart rate, blood pressure, spine curvature, history of falls, diagnosis of medical conditions, and other similar data, as described herein. The fall inference can be generated as a fall inference score (e.g., a numerical score) and/or a classification (e.g., high risk, medium risk, and low risk). The control system 1210 can make use of the fall inference directly (e.g., to actuate actuatable element 1266 or present a sound on speaker 221 of FIG. 1). Additionally, or alternatively, the control system 1210 can send the fall inference information to the EMR system 1260, such as for display on the dashboard display 1262. In some cases, the sensor(s) 1250 can identify an actual fall event (e.g., a resident has actually fallen), which information can be received by the control system 1210 to inform its analysis of the sensor data and generation of future fall inferences, as well as to relay to the EMR system 1260 to store in the EMR database and/or display on the dashboard display 1262.

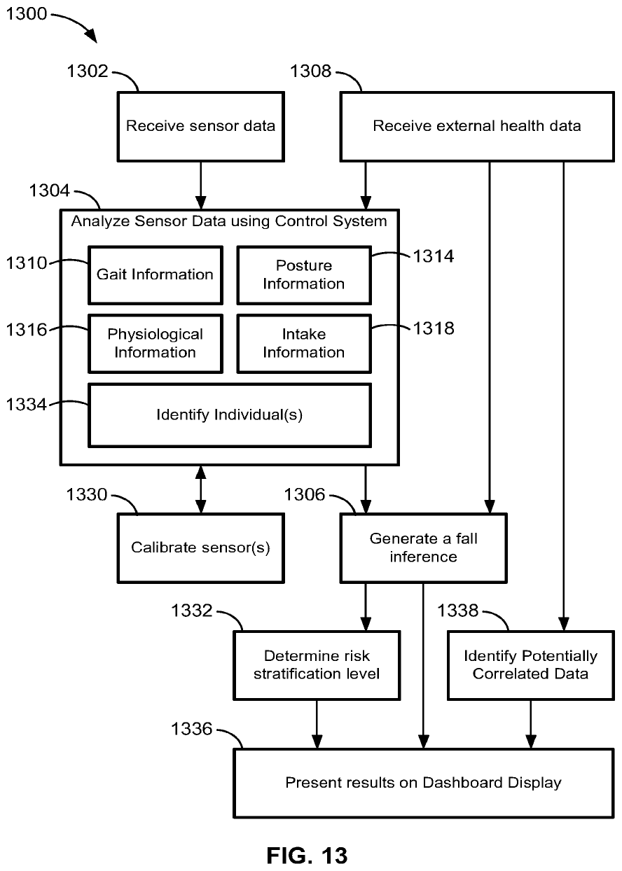

FIG. 13 is a flowchart depicting a process 1300 for determining a falling inference, according to some aspects of the present disclosure. Sensor data (e.g., from the one or more sensor(s) 250 of FIG. 1) can be used by a control system (e.g., control system 110 of FIG. 1) to determine information about a resident (e.g., resident 20 of FIGS. 2-7), which can be used to make a determination about whether or not the resident has fallen and/or whether or not the resident is likely to fall.

At block 1302, sensor data is received from one or more sensors. The sensor data includes data about a resident and/or an environment in which the resident is located. At block 1304, the sensor data is analyzed using the control system. At block 1306, the analyzed sensor data can be used to generate a fall inference, such as whether or not the resident has fallen and/or a likelihood that the resident will fall. While depicted as two separate blocks, in some cases block 1304 and block 1306 can be combined.

Generating the fall inference at block 1306 can include using the analyzed sensor data from block 1304, as described in further detail herein. This analyzed sensor data can include various information in the form of analyzed data, classifications, inferences, and/or scores. In some cases, generating the fall inference at block 1306 can include applying an algorithm to a set of inputs in the form of scores in order to generate a fall inference. The fall inference can be a numerical score, a classification, and/or other type of output. In some cases, the fall inference can include additional information associated with a predicted fall, such as time information (e.g., an exact time window or a general time of day), location information (e.g., near the common room), activity information (e.g., while getting out of bed), an activity (such as an activity with which the resident is engaged) associated with when the fall is predicted to occur (e.g., getting out of bed), or any other information associated with an increased likelihood of falling. In some cases, the algorithm used to generate the fall inference can be a weighted algorithm, applying weights to the various inputs received from the analyzed sensor data and/or from external health data. The various techniques for generating the fall inference are described herein, including with reference to the types of data collected and information analyzed at block 1304.

In some cases, external health data (e.g., EMR data) is optionally received at block 1308. In some cases, generating the fall inference 1306 can optionally include using the external health data and the analyzed sensor data to generate the fall inference. In some cases, analyzing the sensor data at block 1304 can optionally include using the external historical data to facilitate analyzing or interpreting the sensor data. In some implementations, the control system can receive trend data from external sources, such as electronic medical record (EMR) databases, electronic health record (EHR) databases, or other medical or health-related databases. The control system can consider and process data related to a multitude of physiological, movement, and environmental factors, optionally including subjective caregiver notes, to determine a root cause analysis of a gait assessment or fall inference of the resident. Such an assessment can be specific to a particular instance or can be related to trends (e.g., trends in predictions or trends in assessed data). For example, one or more trending parameters may be correlated or likely correlated to a particular gait assessment or fall inference of the resident or ongoing changes in gait assessments or fall inferences of the resident.

Analyzing sensor data at block 1304 can include leveraging the sensor data from one or more sensors to measure, detect, calculate, infer, or otherwise determine information about a resident (e.g., resident 20 of FIGS. 2-7) and/or the environment in which a resident is located. Analyzing sensor data at block 1304 can include any combination of elements that may be helpful in generating the fall inference at block 1306. Some example elements are described with reference to FIG. 13, although in some cases process 1300 will include different elements and/or different combinations of elements.

At block 1310, gait information can be determined. In some cases, one or more sensor(s) generate data that can be processed by the control system to detect one or more aspects and/or parameters of a gait of a resident. Specifically, in some implementations of the present disclosure, the one or more sensor(s) generate data that can be processed by the control system to determine a speed of movement of a resident, an amount of movement of a resident, one or more vitals of the resident (e.g., heart rate, blood pressure, temperature, etc.), a particular position of a resident, a particular movement of a resident, and/or a particular state in which the resident is found. In some cases, the control system can apply an algorithm to incoming data from a sensor (e.g., an ultra-wide band (UWB)-based sensor, an infrared (IR)-based sensor, or a frequency modulated continuous wave (FMCW) sensor) to identify the position of a resident as a location in an indoor space.

In some cases, the sensor data can be analyzed to determine location of the resident in a room, which can be used over time to determine the speed of movement and changes in speed of movement over time. In some cases, if the speed of movement drops below a threshold amount (e.g., 1 m/s for walking speeds) for a certain period of time or length of walk (e.g., 3 meters), the control system can infer that resident may have an increased likelihood of falling. This inference may be made because such changes in speed of movement can be an identifier of fall risk due to changes in physiological capability (e.g., strength and balance) and indication of increased carefulness of the resident (e.g., due to an actual or perceived self-identified risk of falling). Likewise, the sensor data can be analyzed to determine variability in the speed of movement over time (e.g., the number and intensity of changes in speed of movement over a duration). Increased variability in speed of movement (e.g., walking speed) can be indicative of unsteady walking patterns and potential physical decline, which can be indicative of a fall risk.

In some implementations of the present disclosure, the sensor generates data associated with the gait of the resident over time. Such historical data can be stored in an external health database (e.g., received at block 1308) or otherwise. Such data can be processed by the control system for use in predicting if the resident is likely to fall. For example, changes in one or more aspects and/or parameters of the gait of the resident over a period of time (e.g., one hour, five hours, one day, one week, one moth, one year, etc.) can be analyzed to use in a prediction that the resident is likely to fall within a certain amount of time (e.g., within one week, two weeks, three weeks, etc.). Similarly, the sensor is able to generate data that can be processed by the control system to determine that the resident is in the process of falling (e.g., after bumping into the static object).

In some cases, the control system can identify certain body parts of the resident, such as the resident's head (e.g., by using estimated height information and location of sensed motion in space), then use the relative movement of the body parts to help infer whether the resident is falling or likely to fall. For example, an algorithm can determine the amount swaying and/or wobbling side to side and/or back to front of a body part of the resident (e.g., the head of the resident) to assess balance of the resident. Sway can be measured as a distance from an average position of the head in a left-to-right and/or back-to-front motion. A balance score can be assigned according to how much sway is detected. In some cases, generating the fall inference at block 1306 can use the balance score. In some cases, the amount of variability in balance over time can be given a unique score (e.g., a balance variability score) and can be used to generate a fall inference (e.g., a predicted likelihood of a future fall) at block 1306.

In some implementations of the present disclosure, the sensor generates data that can be processed by the control system to determine how the resident's foot/toes/heel are picked up and placed back down while walking. The control system can consider such generated data to determine a specific gait of the resident, which can be indicative of a future likelihood of falling. Thus, the control system can use the determined gait of the resident in generating a fall inference at block 1306.

According to some implementations of the present disclosure, the sensor generates data that is processed by the control system to determine a step height for the resident. The step height can be measured from a floor to a bottom of a heel and/or toe of the resident, from the floor to a knee of the resident, or both. The measurement of step height can be monitored over a period of time by the system and changes in the step height as compared to historical step height data for the resident (e.g., historical average step height, etc.) can indicate deterioration of the gait of the resident and be used to predict an impending fall and/or to determine a risk of fall for the resident.

According to some implementations of the present disclosure, the sensor generates data that can be processed by the control system to determine an amount or measure of swaying of the resident. In some such implementations, a velocity and/or speed of the swaying is determined by the control system. The swaying and its velocity/speed can be measured while the resident is standing, while the resident is walking, while the resident is running, or a combination thereof. The measurement of swaying and/or its velocity/speed can be used to predict an impending fall and/or to determine a risk of fall for the resident.

The control system is also able to analyze the data generated by the sensor to determine or otherwise observe a shortening of a stride of the resident when the resident walks or runs. The control system is also able to analyze the generated data from the sensor to determine an amount of time it took the resident to go from a first location to a second location, an amount of time it took the resident to get out of a chair, an amount of time it took the resident to ascend from a couch, or an average for any of these types of activities over a period of time. The control system is further able to analyze the data to determine whether the amount of time for the resident to complete one or more of the aforementioned activities has increased (e.g., indicating the resident is more likely to fall) or decreased (e.g., indicating the resident is improving and less likely to fall) over time.

In some cases, gait information can include pathway information of a resident moving through the facility. Detection of a resident moving in a straight line through a defined space (e.g. room) to an objective (e.g. desired location, chair, etc.) can be indicative of good, steady, confident walking, whereas movement of the resident around the edges of a defined space (e.g., room) to reach an objective can indicate that the resident is holding on to walls or furniture to assist with walking, which can be an indicator of a fall risk. The pattern of walking and/or deviation from a normal pattern of walking can be indicative of a fall risk. The pathway information can be given a pathway score, which can be used in generating the fall inference at block 1306. For example, as deviations from the resident's normal walking patterns increase, the pathway score can increase.

In some cases, gait information can include touring information. Touring information can include information about a resident leaving his or her room (or other designated area) to visit others (e.g., other residents) and other locations, such as other residents' rooms or common rooms. Touring information can include information about what rooms are visited, the duration in rooms, the number of visits, and other such data. Touring information can also include trips to bathrooms, kitchens, or the like. For example, an increase or decrease in the number of trips to a bathroom can be indicative of certain medical issues (e.g., constipation, urinary tract infection, and the like). In another example, a decrease in the number of trips to a kitchen can be indicative of loss of appetite.

Determining gait information at block 1310 can include determining one or more gait scores associated with any of the gait information, such as a score associated with speed of movement changes, a score associated with changes in parameters of the gait as compared to historical data, a score associated with a resident's location in a space, and the like. The gait score(s) can be used in generating the fall inference at block 1306.

In some cases, determining gait information at block 1310 can optionally include using information from one or more other blocks within block 1304, such as blocks 1314, 1316, and/or 1318. For example, determining gait information at block 1310 can include using posture information determined at block 1314 (e.g., using an identified posture of a resident to help interpret sensor data into gait information).

At block 1314, posture information can be determined. As used herein, the term posture is inclusive, as appropriate, of an overall body posture (e.g., whether an individual is lying, sitting, standing, or the like), as well as body-part postures (e.g., curved back, tipped head, bent legs, straight arm, and the like). In some implementations of the present disclosure, the sensor generates data that can be processed by the control system to determine an average time in bed for the resident, an average time sitting for the resident, an average time standing for the resident, an average time moving of the resident, and a ratio of time spent in bed, sitting upright, etc. Such data can also be used to determine one or more aspects and/or parameters of the gait of the resident and/or to predict when the resident might fall and/or if the resident is currently falling. In some cases, a ratio of sedentary time versus ambulatory time can be tracked over time. An increase in this ratio can be indicative of a fall risk, and can be indicative of decreased agility, energy, and physical strength. In some cases, an increase in sedentary time can be indicative of certain mental health issues, such as depression. In some cases, a sedentary-ambulatory score can be generated, which can be used to generate the fall inference at block 1306.

In some implementations of the present disclosure, the sensor generates data that can be processed by the control system to determine one or more aspects and/or parameters of a posture of the resident. Such posture aspects and/or parameters can include a characterization of a position of one or more portion of the resident (e.g., curved back, tipped head, bent legs, straight arm, etc., or any combination thereof), a current or average movement amount for one or more portions of the body of the resident, a current or average state for one or more portions of the body of the resident, or any combination thereof. For example, the generated data can be processed by the control system to determine whether the resident is lying down in an object (e.g., a bed) or on a surface (e.g., a floor), whether the resident is sitting (e.g., in a chair, on a table, on the floor, etc.), whether the resident is moving (e.g., walking, running, being pushed in a wheel chair, etc.), whether the resident is about to fall, whether the resident has tripped and/or stumbled, whether the resident is sleeping, whether the resident is in the process of standing from a seated position, whether the resident is in the process of sitting from a standing position, etc., or any combination thereof. Such data can also be used to determine one or more aspects and/or parameters of the gait of the resident and/or to predict when the resident might fall and/or if the resident is currently falling.

In some cases, the sensor can use posture and position detection to determine the number of sit-to-stand attempts required for a resident to stand up completely. An increase in the number of sit-to-stand attempts can be indicative of loss of strength or balance and can be indicative of a fall risk. In an example, a sensor can identify the height of the resident's head and identify repeated bobbing up and down as unsuccessful sit-to-stand attempts.

In some implementations of the present disclosure, the sensor generates data that can be processed by the control system to determine if the resident is swaying while standing. The control system can also determine, based on the generated data, the velocity of swaying to determine fall risk of the resident.

In some implementations of the present disclosure, the sensor generates data that can be processed by the control system to determine a level of mobility of the resident. For example, the generated data can be processed to determine an assessment on how the individual bends at the knee, at the hip, etc., to determine mobility and/or imbalance. The control system can determine the mobility and imbalance as a proxy for fall risk of the resident.

In some implementations of the present disclosure, the sensor generates data that can be processed by the control system to determine a current or average state time the resident has spent on the floor after a fall. In some implementations of the present disclosure, the sensor generates data that can be processed by the control system to determine movements of the resident in the current or average state time, post-fall, to characterize how the resident attempts to get up and re-balanced.

In some cases, information about posture of the resident's spine can be determined from the sensor data when combined with external health data from block 1308. For example, medical notes and/or other records about the resident's spine curvature can be used to inform the analysis of the sensor data when determining the resident's posture. In an example, the distance from the back of the resident's head to a wall or to an imaginary vertical line extending from the base of the spine can be tracked. An increase in this distance (e.g., more spine curvature) can be indicative of a fall risk. While described as an example for spine curvature, similar techniques can be applied to any other information being analyzed during block 1304.

In some cases, sensor data can be used to detect and quantify involuntary movements of a resident, such as tremors in hands and arms. Increases in such involuntary movements can be indicative of certain conditions, such as Parkinson's disease, and can be indicative of a fall risk.

Determining posture information at block 1314 can include determining one or more posture scores associated with any of the posture information, such as a score associated with average sitting time, a score associated with mobility level, a score associated with sway, and the like. The posture score(s) can be used in generating the fall inference at block 1306.

At block 1316, physiological information (e.g., such as heart-related, respiration-related, and/or temperature-related information) can be determined. The physiological information can include information based on measurements of the resident's physiological functions, such as breathing, circulation, temperature, and others. In some implementations of the present disclosure, the sensor generates data that can be processed by the control system to determine an average breathing/respiration rate of the resident, an average heart rate of the resident, an average blood pressure of the resident, an average temperature (e.g., core, surface, mouth, rectal, etc.) of the resident, or any combination thereof. Such data can also be used to determine one or more aspects and/or parameters of the gait of the resident and/or to predict when the resident might fall and/or if the resident is currently falling. In some cases, physiological information can also provide an indication that a resident may have a fever or may be otherwise infected. For example, changes in heart rate, breathing rate, and/or temperature can be used to flag a potential health condition.

In some cases, physiological information at block 1316 is respiration-related and can be used to detect, monitor, and/or predict respiration-related disorders such as Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), Chest wall disorders, and Severe acute respiratory syndrome (SARS) related coronaviruses such as coronavirus disease 2019 (COVID-19) caused by severe acute respiratory syndrome coronavirus 2 (SARS-CoV-2). In respect of COVID-19 in particular, common symptoms can include fever, cough, fatigue, shortness of breath, and loss of smell and taste. The disease is typically spread during close contact, often by small droplets produced during coughing, sneezing, or talking. Management of the disease currently includes the treatment of symptoms, supportive care, isolation, and experimental measures. During recovery from COVID-19, including following discharge from a hospital setting, it is beneficial to monitor patient vital signs, including coughing events, to track a patient's recovery and to provide an alert if the patient's condition deteriorates, which might require further medical intervention. Examples and details on identifying and monitoring respiration events, e.g. coughing events, can be found in WO2020/104465A2 (e.g., paragraphs [0054]-[0062], [0323]-[0408] and [0647]-[0694]), which is hereby incorporated by reference herein in its entirety. In some embodiments, the present technology includes a system or method for monitoring the physiological condition of a person, comprising: identifying coughing by a person by (i) accessing a passive and/or active signal generated by non-contact sensing in a vicinity of the person, the signal representing information detected by a non-contact sensor(s), (ii) deriving one or more cough related features from the signal, and (iii) classifying, or transmitting for classification, the one or more cough related features to generate an indication of one or more events of coughing by the person. The system or method may further comprise receiving physiological data associated with one or more physiological parameters of the person. The physiological data may be generated via one or more contact and/or non-contact sensors, may be provided as subjective data, and/or may be accessed from a health or medical record. Such physiological data can include blood pressure, temperature, respiration rate, SpO2 (oxygen saturation) level, heart rate, change or loss of perception of taste and/or smell, respiration effort such as shortness of breath and/or difficulty in breathing, gastrointestinal disorder such as nausea, constipation and/or diarrhea, skin appearance such as rash or markings on toe(s) (e.g. “COVID toe”), and any combination thereof. Certain aspects and features of the present disclosure may make use of movements of the person as they cough, as detected by one or more motion sensors (such as RF sensor, sonar sensor (such as a microphone and speaker), LiDAR sensor, accelerometer, and others such as described herein), to detect, monitor, and/or identify the coughing events. In some embodiments, a detected passive signal (such as an acoustic sound detected by a microphone) of coughing may be combined with the movements of the person as they cough as detected by one or more motions sensors (such as RF sensor, sonar sensor (such as a microphone and speaker), LiDAR sensor, accelerometer, and others such as described herein) to estimate physiological parameters, such as chest wall dynamics, and further characterize the nature of the cough. Examples of physiological parameters include breathing rate, heart rate, blood pressure, cough signature, wheeze, snore, sleep disordered breathing, Cheyne-Stokes Respiration, sleep condition, and electrodermal response. In some cases, any one or more of inspiration time, expiration time, a ratio of inspiration-to-expiration time, and respiratory waveform shape may be estimated. Optionally, the physiological parameter may comprise respiratory rate, and trend monitoring of respiratory rate variability (RRV) may be applied. In some cases, the physiological parameter may comprise heart rate, and trend monitoring of heart rate variability (HRV) may be applied. Thus, the methodologies described herein may detect, monitor, and/or predict respiration events with passive sensing technologies (such as via acoustic sound sensing) and/or one or more active sensing technologies (e.g., RADAR and/or SONAR sensing). For example, the methodologies described may be executed by one or more processors such as (i) with an application on a processing or computing device, such as a mobile phone (e.g., smartphone), smart speaker, a smart TV, a smart watch, or tablet computer, that may be configured with a speaker and microphone such that the processing of the application implements a synergistic fusion of passive sensing (acoustic breathing related sound monitoring) and active acoustic sensing (e.g., SONAR such as with ultrasonic sensing), (ii) on a dedicated hardware device implemented as a radio frequency (RF) sensor (e.g., RADAR) and a microphone, (iii) on a dedicated hardware device implemented as an RF sensor without audio; and/or (iv) on a dedicated hardware device implemented as an active acoustic sensing device without RF sensing. Other combinations of such devices will be recognized in relation to the details of the present disclosure. Thus, such devices may be independent or work cooperatively to implement the passive and active sensing with any of the detection/monitoring methodologies described herein.

In some cases, physiological information at block 1316 can be used to predict a future action of the resident. For example, increased breathing rate while the resident is sleeping in a bed can be an indication that the resident is waking up and may likely wish to exit the bed, which may be an indicator for an imminent fall risk.

Determining physiological information at block 1316 can include determining one or more physiology scores associated with any of the physiological information, such as a score associated with breathing rate, a score associated with heart rate variability, a score associated with average temperature, and the like. The physiology score(s) can be used in generating the fall inference at block 1306.

At block 1318, resident intake information can be determined. Intake information can include information about what medications, foods, or drinks a resident has consumed or has otherwise been introduced into the resident's body. For example, consumption of certain medications or foods, or lack thereof, may be used to infer that the resident may become dizzy or lightheaded, which may increase a likelihood of a fall. In some implementations, the control system can determine a multi-factorial characterization of the hydration of the resident. This characterization can be determined by assessing a location of the resident, a body movement, and an arm movement of the resident. In some cases, the hydration of a resident can be used to help infer a resident's destination when the resident begins to move. For example, the sensor can generate data that can be processed by the control system to determine that the resident is advancing towards a restroom, a sink, or a refrigerator.

Determining intake information at block 1318 can include determining one or more intake scores associated with any of the intake information, such as a score associated with hydration, a score associated with micturition, a score associated with medication intake, and the like. The intake score(s) can be used in generating the fall inference at block 1306

In some optional cases, at block 1334, one or more individual(s) can be identified using the sensor data. Identifying an individual at block 1334 can be a part of analyzing the sensor data at block 1304, although that need not always be the case. In some cases, identifying an individual can occur as a block separate from analyzing sensor data at block 1304 and/or can occur as part of generating the fall inference at block 1306. Identifying an individual can involve associating current sensor data with a unique identifier (e.g., an identification number), associated with an individual. Identifying an individual does not necessarily require determining any personally identifiable information or personal health information about the individual, although in some cases a unique identification number associated with an individual can be used to link the sensor data and fall inferences with records in an EMR system. The purpose of identifying individuals at block 1334 can include filtering out extraneous data so that only data associated with a single individual is used to determine the fall inference for that individual. For example, identifying individuals at block 1334 can avoid false inferences if someone other than the resident in question is moving around in the resident's room in a way that would indicate a fall risk or would indicate no fall risk. Identifying individuals at block 1334 can make use of any of the information determined as part of analyzing the sensor data at block 1304, such as gait information from block 1310, posture information from block 1314, physiological information from block 1316, or intake information from block 1318, which, for example, uniquely identify the resident in the context of the environment (e.g., of a house, hospital, or other facility) in which the resident resides.

In some implementations, the system continues to monitor the speed of movement of the resident over a period of time, the gait of the resident over a period of time, a balance of the resident over a period of time. Furthermore, in some implementations of the present disclosure, the system is able to monitor multiple residents. In such a configuration, a unique signature can be provided and/or determined to detect movements of each resident to generate a profile for each resident. Such a unique signature can be based at least in part on a heart rate signature, a respiration signature, a temperature signature, a body profile, a face profile, one or more facial or body features, an eye signature, etc., or any combination thereof.

In some implementations of the present disclosure, the sensor generates data that can be processed by the control system to determine characteristics about each individual person. The information generated from the sensor data can include characteristics such as height, weight, breath rate, heart rate, and other biometrics used to distinguish two people from each other. Data from the EMR system can facilitate distinguishing individuals. In some implementations of the present disclosure, the sensor generates data that can be processed by the control system to determine personal characteristics, such as height, weight, breath rate, heart rate to track, detect, and distinguish people moving from one room to another. The sensor data and processed data can include data from multiple people simultaneously congregated in common areas.

In some cases, analyzing sensor data at block 1304 can include determining environmental information. Determining environmental information can make use of sensor data received at block 1302 that is associated with the environment itself. For example, such data can include temperature data (e.g., ambient air temperature data), humidity data, light level data (e.g., environmental luminance), and other such data about the environment. Determining the environmental information can include determining ambient temperature information, humidity information, light level information, and the like. The environmental information can be used in generating the fall inference at block 1306. As an example, high ambient temperature (e.g., higher than usual), high humidity (e.g., higher than usual), and/or low light levels may be indicative of an increased fall risk as compared to nominal ambient temperature, nominal humidity, and higher light levels. Thus, all other information being equal, a resident located in a room may have a higher fall risk if the room is dark (e.g., the room has low light levels) than if the room is well-lit (e.g., the room has higher light levels). In some cases, scores for environmental information can be generated (e.g., an overall environmental score and/or individual scores for temperature, humidity, light level, and the like). Such scores can be used in generating the fall inference at block 1306.

In some cases, at optional block 1330, the one or more sensors can be calibrated using the analyzed sensor data from block 1304. Calibration can include overall system calibration (e.g., to calibrate sensors installed in a facility) as well as calibration for an individual (e.g., to calibrate models used to analyze, and generate inferences from, sensor data). Calibration can occur for a period of time to build up baseline data. In some cases, individual calibration can also be facilitated by received external health data from block 1308.

In some implementations of the present disclosure, the sensor generates data that can be processed by the control system to determine a capacity to use other, more mobile people, other than the resident, to configure the sensor(s) for each individual room installation. This installation allows calibration of the sensor and the control system in, for example, under 24 hours.

In some implementations, the system can also include a sensor marker (e.g. a sticker, or patch), worn or carried by the resident that can synchronize with the sensor and/or control system for a more in-depth read of physiological data (e.g., via collecting physiological data directly from sensors on a resident rather than relying on sensors remote from the resident) and completion of an admission based fall risk assessment upon entry to a nursing home. In some cases, such an admission based fall risk assessment can be automatically completed using historical data and/or EMR data. The markers allow for immediate assessment, not needing calibration time.

In an example, the received sensor data at block 1302 can be analyzed at block 1304 to determine data related to a resident's walking velocity, data related to pathways traversed by the resident, data related to the sway of the resident, data related to the overall activity level of the resident, or combinations thereof. In this example, the fall inference generated at block 1306 can be based on a weighted formula. In this example, a rule for each type of data analyzed at block 1304 can be applied to the relevant data to generate a score for that type of data. For example, walking velocity above 1 m/s may be considered normal and be assigned a score of 1, walking velocity above 0.4 m/s and at or below 1 m/s may be considered abnormal and assigned a score of 2, and walking velocity at or below 0.4 m/s may be considered frail and assigned a score of 3. Each score can be assigned a weighting according to its strength of correlation to risk of falling. Then, each weighted score can be added together to calculate a fall risk score associated with the sensor data (e.g., a sensor-derived fall risk score).

In another example, the received sensor data at block 1302 can be analyzed at block 1304 to determine data related to a resident's actual fall events, data related to a resident's walking velocity, data related to pathways traversed by the resident, data related to the sway of the resident, data related to the overall activity level of the resident, or combinations thereof. In this example, the fall inference generated at block 1306 can be based on machine learning. When machine learning is used, the machine learning algorithm can correlate each parameter (e.g., each type of analyzed data) against actual falls and other behavioral events. The machine learning algorithm can learn over time by comparing the relevant fall risk parameters to actual fall events. Thus, the trained machine learning algorithm can accept the sensor data and/or analyzed sensor data as input and then output an appropriate fall risk score associated with the sensor data (e.g., a sensor-derived fall risk score).

As disclosed herein, generating the fall inference at block 1306 can make use of external health data received at block 1308. In some cases, the external health data can include a health-data-derived fall risk score or data usable to generate a health-data-derived fall risk score. A health-data-derived fall risk score can be a fall risk score that is derived from health data and not based solely on the sensor data received at block 1302. For example, a system can use health data that includes data related to a resident's blood pressure, medication usage, history of fall events, progression of degenerative diseases (e.g., Parkinson's disease and/or dementia), and the like, to calculate the health-data-derived fall risk of an individual over time. The health-data-derived fall risk can be calculated by applying the health data to a machine learning algorithm, although that need not always be the case.

In some cases, generating the fall inference at block 1306 can include making use of a sensor-derived fall risk score (e.g., a fall risk score derived from the sensor data received at block 1302) and a health-data-derived fall risk score. In some cases, generating the fall inference at block 1306 can include outputting an average of, a highest of, or a lowest of the sensor-derived fall risk score and the health-data-derived fall risk score.

In some cases, a sensor-derived fall risk score can be provided to a health records system (e.g., EMR system 1260 of FIG. 12) and can be used as an input to the formula and/or machine learning algorithm used to generate the health-data-derived fall risk score. In some cases, sensor data (e.g., raw sensor data, analyzed sensor data, and/or weighted sensor data) can be provided to a health records system (e.g., EMR system 1260 of FIG. 12) and can be used as input(s) to the formula and/or machine learning algorithm used to generate the health-data-derived fall risk score. In these sets of cases, the sensor-derived fall risk score and/or different types of sensor data can be stored in a network-accessible (e.g., cloud-based) server, optionally processed (e.g., to generate individual scores and/or weighted scores for different types of sensor data), and provided to a health records system (e.g., EMR system 1260 of FIG. 12) using individual APIs. Thus, the sensor-derived fall risk score and/or the individual weighted scores can be combined with and/or used to generate the health-data-derived fall risk score.

In some cases, a health-data-derived fall risk score can be received at block 1308 and used as an input to the formula and/or machine learning algorithm used to generate the sensor-derived fall risk score.

In some cases, generating the fall inference at block 1306 can include generating a fall risk score that is a combined fall risk score. In some cases, a combined fall risk score can be generated by using sensor data (e.g., sensor data from block 1302 and/or analyzed sensor data from block 1304) and external health data from block 1308 as inputs to a machine learning algorithm that outputs the combined fall risk score. In some cases, a combined fall risk score can be generated by using a sensor-derived fall risk score as an input to a machine learning algorithm being run on external health data. In some cases, a combined fall risk score can be generated by using a health-data-derived fall risk score as an input to a machine learning algorithm being run on sensor data.

In some cases, at optional block 1332, a risk stratification level can be determined. The control system can consider and process data related to a risk stratification model (e.g., a traffic light based risk stratification model) of multiple residents across a facility. For example, a gait analysis and/or fall inference can be determined for multiple residents. Determining a risk stratification level for each resident can include comparing scores associated with each resident's gait analysis and/or fall inference to a set of threshold levels to assign each resident to one of the levels of the risk stratification model. For example, in a traffic light model, the model would include at least two thresholds, such that those with a risk score above the first (e.g., highest) threshold would be considered “high risk,” those with a risk score above the second threshold and up to the first threshold would be considered “medium risk,” and those with a risk score at or below the second threshold would be considered “low risk.” Other numbers of levels can be used. In some cases, any of the thresholds used can be dynamically adjusted based on the various scores of the residents in a facility. For example, if too many residents are deemed “high risk,” the model can dynamically adjust so that the first threshold is raised until the number of residents deemed “high risk” reaches a preset maximum.

In some cases, determining risk stratification levels at block 1332 can include presenting information about the residents in a facility in a ranked list based on each resident's gait analysis and/or fall inference (e.g., a fall inference score or classification).

The use of a risk stratification model can ensure each resident receives appropriate fall prevention, fall detection, and fall mitigation services. The control system is able to efficiently determine how to provide to each resident the necessary service without compromising the efficiency of the system as a whole.

In some cases, potentially correlated data can be identified at optional block 1338. Potentially correlated data can include any data, such as sensor data or external health data, that may be correlated with the fall inference generated at block 1306.

In some cases, gait analysis and/or fall inference (e.g., fall inference scores) can be tracked over time. In some cases, potentially correlated data can be presented alongside gait analysis and/or fall inference. For example, a resident with a consistent fall inference may experience a sudden increase in fall inference (e.g., indicative of an increase as a fall risk) on a particular day at a particular time. Potentially correlated data can be identified from any available data source (e.g., sensor data, analyzed sensor data, and/or external health data) based on timestamp information (e.g., by using timestamped data to identify potentially correlated parameters or events that may have led to a particular gait analysis and/or fall inference). For example, an indication in an EMR database that the resident had been prescribed new medication earlier that day (or week, month, etc.) can be presented as potentially correlated with the sudden change in fall inference. Thus, practitioners and caregivers can quickly investigate how various changes affect each resident's fall risk and use that information to tailor further care of the resident. In some cases, the potentially correlated data can be indicative of a cause of the increased fall risk.

In some cases, potentially correlated data can be determined for a fall event. When a fall event has been detected (e.g., from a sensor) or otherwise identified (e.g., from EMR data), the control system can mine through its available data (e.g., sensor data and/or EMR data) to identify an activity or change that may have led to the fall event. For example, after a fall has occurred and been identified, the control system may identify EMR data indicating the resident has been prescribed a medication, but the recent sensor data prior to the fall is indicative that the resident did not take the prescribed medication and did not hydrate sufficiently prior to the fall event. Thus, practitioners and/or caregivers can be provided with this potentially correlated data (e.g., on a dashboard display like dashboard display 1262 of FIG. 12) in order to identify failings in treatment and/or identify ways to improve ongoing treatment of this and/or other residents. In some cases, the potentially correlated data can be indicative of the success of certain interventions or therapies in improving a resident's fall risk score. Continuous updates to fall risk score along with providing potentially correlated data can be used to show progress as a holistic management system.

In some cases, analyzing the sensor data at block 1304 and/or generating the fall inference at block 1306 can make use of external health data received at block 1308, such as from an EMR system (e.g., EMR system 1260 of FIG. 12). The external health data as described with reference to process 1300 of FIG. 13 is inclusive of medical data, as well as relevant related data about the resident, such as demographic data and other data.

In some implementations, the control system is configured to receive and process feedback input related to the demographic and location data associated with a resident. The feedback can also be received from EMR databases (e.g., an EMR system) to aid in identifying the resident and/or an expected location (e.g., room number) of the resident that had the fall and/or for which a likelihood of a fall was determined.

The control system can be configured to generate configurable trend reporting of residents to determine fall risks. The control system is able to transmit a generated trend data to caregivers and clinical staff, such as via EMR databases (e.g., the EMR system).

In some cases, EMR data can be accessed to identify if a particular resident has a particular diagnosis or certain medical history. Based on this information, the control system can adjust the analysis of sensor data and/or the generation of a fall inference. In some cases, the control system can also identify sensor data usable to support or refute existing EMR data (e.g., data supporting or refuting a diagnosis or suspected diagnosis).

In an example case, EMR data indicating a diagnosis of Parkinson's disease can be used to modify how one or more gait scores and/or one or more posture scores are generated. In another example, EMR data indicating a diagnosis of dementia can be used to modify how pathway information or touring information is assessed (e.g., placing more weight on the resident's deviations from typical movement pathways). In an example, EMR data indicating a history of mental illness and/or psychological health issues (e.g., depression and dementia) can be indicative of an increased fall risk.

In an example case, EMR data indicating a diagnosis of a condition affecting the white matter of the brain, such as white matter disease or microvascular ischemic disease, can be used to modify how one or more gait scores and/or one or more posture scores are generated (e.g., gait, balance, sway, movement, and/or pathway scores). Likewise, changes in walking behavior, sway, balance, mood, mental health, reduced movement, bathroom usage, increased number of falls identified by the sensor(s), and the like can be used to indicated the onset of a condition affecting the white matter of the brain, such as white matter disease.

In an example, EMR data indicative of prolonged hypertension or high heart rate can be indicative of a fall risk and used to inform generation of the fall inference. In an example, EMR data about a resident's age can be indicative of a fall risk (e.g., older individuals may be more likely to be at a risk of falling).

In an example, EMR data indicating a history of falls as recorded by practitioners or caregivers, or as self-reported, can be indicative of an increased fall risk. In an example, EMR data about the length of time a resident spent recovering from a past fall (e.g., rehabilitation or time at a nursing facility) may be indicative of a fall risk (e.g., longer rehabilitation time can be indicative of an increased fall risk). Similarly, length of time spent in acute care can be indicative of a fall risk (e.g., longer time in acute time can be indicative of an increased fall risk).

In an example, EMR data about a resident's muscular skeletal performance (e.g., leg and/or knee strength during rehabilitation or after rehabilitation) can be used to inform generation of the fall inference (e.g., poor leg and/or knee strength can be indicative of an increased fall risk).

In an example, EMR data about medication intake can be indicative of a fall risk (e.g., the use of psychotropic drugs can lead to an increased fall risk).

In some cases, the external health data received at block 1308 is received from a live (e.g., real-time) EMR system, such as one currently being used to manage care of the resident. In such cases, the use of live data can help identify sudden changes or deviations from normal health data (e.g., blood pressure, medical prescriptions, change in care settings, and the like), which can be used to inform analysis of sensor data and/or generation of the fall inference. For example, a resident who was recently placed on new medication or a new medication regimen may exhibit an altered gait which is expected due to the new medication or new medication regimen, and since the control system received the dynamic update about the resident's new medication from block 1308, the control system can use that information to adjust the scoring and/or fall inference generation accordingly (e.g., if the altered gait were not expected, it might have otherwise been indicative of a higher risk of imminent fall). In some cases, the control system may be connected (e.g., directly or indirectly, such as via an EMR system) to an electronic pillbox and to prescription information, such as to determine if new or revised medication has been prescribed and whether or not the user has taken that medication. Thus, the system can check for pharmacological reasons (e.g., potentially correlative data) why a resident's risk of falling may be higher or lower than before. As an example, apart from (or including) utilizing EMR data, it is possible to use other real-time patient medical adherence tools or their live status as real-time input to a fall risk and fall prediction classifier. For example, such data sources can include pill adherence, injection adherence (e.g., a smart, connected sharpie bins), inhalers adherence, and the like.

In some implementations of the present disclosure, the system (e.g., system 100 of FIG. 1) processes at least a portion of the generated data from the sensor in a cloud system and provides alerts, a fall risk, trend data, a software platform, an app based platform, or any combination thereof. In some such implementations, the system can generate a fall detection alert. The fall detection alert can be provided as an audible voice command (e.g., via speaker 221 shown in FIG. 1) and/or be transmitted to a portable speaker (e.g., a walkie-talkie) and/or a pager or mobile phone.

Certain blocks of process 1300 can be performed using algorithms in order to generate scores, classifications, inferences, and/or other results. These algorithms can be weighted algorithms. In some cases, such algorithms can make use of machine learning to improve the accuracy of a score, classification, inference, and/or other result. Such machine learning can be trained using a resident's data (e.g., historical sensor data and health information, as available) and/or data from a cohort of residents (e.g., multiple individuals associated with a particular facility). The machine learning training can help identify patterns in the various data inputs and correlate them with a likelihood of falling or other suitable information as described herein. Other data analysis techniques can be applied to improve determining the fall inference, such as deep neural network modules, such as a convoluted neural network (CNN) or recurrent neural network (RNN) (e.g., long short-term memory units).

In some cases, analyzing sensor data at block 1304 and/or generating the fall inference at block 1306 can make use of both current sensor data (e.g., live sensor data) and historical sensor data (e.g., data over a certain age, such as 1 hour, 1 day, 1 week, 1 month, 1 year, or any other amount of time). In some cases, the determination of a static object (e.g., static object 275 of FIG. 2) or other objects in an environment can also be considered historical data. The current data and the historical data can be accumulated and/or processed. The control system is configured to train a machine learning algorithm with the historical data. A memory (e.g., memory 114 of FIG. 1) can include machine-readable instructions which include the machine learning algorithm. The machine learning algorithm is configured to receive the current data as an input and determine, as an output, a predicted time and/or a predicted location that the resident will experience a fall (e.g., a fall inference).

At block 1336, results can be presented on a dashboard display. In some cases, presenting results on the dashboard display can include presenting only a fall inference for a resident, such as information related to a fall event or a likelihood of the resident falling (e.g., a fall risk score such as a sensor-derived fall risk score, a health-data-derived fall risk score, or a combined fall risk score). In some cases, presenting the results can further include presenting the results in the form of a risk stratification level or using risk stratification scheme. For example, presenting the results at block 1336 can include displaying the resident's name in the “High” risk category when the fall inference generated at block 1306 is indicated to be in the high risk category at block 1332. In some cases, presenting the results at block 1336 can separately or additionally include presenting potentially correlated data identified at block 1338.

Referring back to FIG. 1, in some implementations of the present disclosure, the system 100 is deployed in one or more diverse settings/environments, such as, for example, a senior living environment, an independent living environment, a nursing home, a retirement homes, a skilled nursing facility, a life plan community, a home health agency, a home (alone or with family members for example), a hospital, etc., or any combination thereof.

In some implementations, the system 100 can track the path of one or more persons (e.g., residents, family members, care providers, nurses, doctors, etc.) in the environment, using one or more of the sensors 250. One approach, using data from one or more of the sensors 250, includes a Time of Arrival (TOA) algorithm and/or a Direction of Arrival (DOA) algorithm. The output of the algorithm(s) can be used to deduce movements of a target (e.g., resident), and track movements of the target (e.g., track movements of a resident over time). The approach can also include tracking one or more biometrics of the moving target (e.g., resident). The algorithms(s) can be trained over time and can learn (i) the usual or more common paths of a resident within an environment, (ii) the typical speed of movement of the resident, (iii) the number of steps covered by the resident with a predetermined amount of time (e.g., per day), or any combination thereof. The number of steps of the resident can be extracted and/or determined based on the repetitive movement detected (such as via a peak and trough search of a 3D spectrogram) along an identified path.

The tracking of the resident by the system 100 can also include analysis of the data from one or more of the sensors 250 to look for and/or detect (i) a relative increase in randomness in paths traversed by the resident, (ii) a relative increase in wobbles (e.g., due to an issue with gait, or the resident is moving in an unusual or distracted/confused manner), or the like. In such implementations, a detected increase in randomness of traversed paths and/or wobbles can be indicative of Alzheimer's, dementia, multiple sclerosis (MS), behavioral disorders, etc. in the resident. This detection could include determining the resident has motor neuropathy conditions where a fall inference can be utilized to help assess the degree of acceleration in muscle weakness and atrophy. For example, as muscles weaken and atrophy, the fall inference of the resident may indicate higher likelihood of falling.

In some implementations, the system 100 can learn about the environment of the resident based on static reflections captured in the data generated by the sensors 250 (e.g., the IRUWB sensor 256). For example, the system 100 can learn of the location of fixed (or seldom moved) objects such as beds, chairs, tables, other furniture and obstacles. Further the system 100 can compare current data with prior data to identify any changes in the location of fixed objects over time (e.g., such as a chair that is moved by, for example, a cleaning person).

In some implementations, the system 100 can detect a resident within a range of one or more of the sensors 250 by monitoring one or more biometrics of the resident. Such biometric monitoring is advantageous as the resident can be detected even if the resident is not moving and/or has not moved for a period of time (e.g., 1 minute, 5 minutes, 10 minutes, 1 hour, etc.). In some such implementations, the system 100 monitors heart rate and/or breathing rate. The system 100 can monitor biometrics for one or more residents and/or other persons at the same time. Such biometrics can be detected and/or monitored using, for example, RADAR, LIDAR, and/or SONAR techniques. Examples and details on monitoring biometrics can be found in WO 2016/170005, WO/2019/122412, and WO/2019/122414, each of which is hereby incorporated by reference herein in its entirety.

In some implementations, the system 100 monitors a temperature and/or heat signature of one or more residents at a distance. As such, the system 100 can track changes and/or movements of the thermal signature (such as by PIR and/or 3D thermal imaging).

The monitoring and/or tracking of one or more biometrics for one or more residents is beneficial when, for example, a resident falls and is unconscious but still breathing. In such an example, the physical characteristics that might be readily tracked when the resident is standing, walking, and/or sitting (e.g., height) are not useful for identification of the resident. Rather, even when lying on the floor/ground, the biometrics of the resident can be registered and/or detected by the system 100 and used for identification purposes.

Further, analysis of the biometric data from one or more of the sensors 250 can be used to identify an increase in heart rate of a resident and shallower and/or faster breathing of a resident. In some implementations, when such characteristics of the biometric data for a resident are detected, for example, following a large movement by the resident and coupled with a change in height and/or location of the biometric source, the system 100 can indicate a potential fall occurred (e.g., the resident fell).

As discussed herein, a fall can occur from a standing or walking position, from a sitting position (such as bed, chair, toilet etc.), from a lying position (e.g., from bed or couch), or any combination thereof.

In some implementations, a fall could be related to paralysis such as due to a seizure or stroke, the resident tripping, falling, falling out of bed, or suffering a blow (such as a hit on the head by an object, such as a falling object), or losing consciousness (e.g., sudden drop in blood pressure, fainting etc.). In most of such implementations, after a fall occurs to a resident, the resident ends up on the floor or ground as a result thereof. In some such implementations, the resident is rendered unable to call for help.

As discussed herein, the system 100 can include more than one of the sensors 250 that are generating data at the same time and/or about the same time that can be analysed by the control system 110. In some such implementations, the system 100 may analyse a first set of data generated by a sensor (e.g., RF sensor 255) (and/or an acoustic sensor including the microphone 254), to detect movement of a resident in a relatively larger space. Then the system 100 may analyse a second set of data generated by a relatively more localised sensor, such as, for example, one or more RF beacons (e.g., Bluetooth, Wi-Fi or cellular) from a smart device (e.g., a mobile phone). Other examples of relatively more localised sensors include a tag (e.g., an RFID tag) on a key ring, a tag on a wallet, a tag attached to clothing of the resident, etc.

In some implementations, if and/or when a resident is identified based on the resident's physiological parameters and/or biometrics, a smart phone associated with the resident can be automatically called by the system 100 and/or patched through to a human monitor. As such, the condition of the resident can be confirmed (e.g., did the resident actually fall or was the system 100 in error).

Relatively more localised sensing discussed herein (such as location, biometrics, and so forth) can be provided by the IRUWB sensor 256, an RF UWB sensor, one or more accelerometers, one or more magnetometers, one or more gyrometers, or any combination thereof. Such sensors can be integrated in a mobile phone, such as via an INFINEON™ chip (e.g., SOLI™ in a GOOGLE™ PIXEL™ phone), similar chips in an APPLE™ IPHONE™ or other system-on-chip solutions.

In some implementations, the system 100 carries out multi modal fusion, such as processing infrared (active, passive, or a combination) or CCTV or other video images from a hallway or common area, then fuses such image(s) with RF sensing in a living area, bedroom, or bathroom/toilet. Thus, information from more than one of the sensors 250 may for used in a variety of ways and/or manners. A plurality of sensors 250 may work in parallel, the data from each one of the sensors 250 being combined and/or merged to obtain information of any events at a predetermined single location. This may be done as a matter of routine, or only in circumstances where for various reasons the data may not be very reliable and using the data from more than one sensor may provide greater certainty of the detected outcome. Alternatively, data from each of the sensors 250 may be used in a sequential manner to identify the events at the same place. For example, the use of a video camera in the visible range can be replaced/complemented by the use of an IR camera or an RF sensor, if, for example, the lights in the room have been switched off. Data from a number of the sensors 250 can also be used sequentially to build a picture of the sequence of events occurring at different places. For example, data from a second sensor placed in a second room may be used to identify the events that have occurred after the subject has left the first room, monitored by a first detector, and entered the second room, monitored by the second detector.

In some implementations, the system 100 uses data generated from one or more of the sensors 250 to determine motion of a resident. The determined motion can be a movement of a chest of the resident due to respiration, a sway motion (e.g., when standing or sitting), a sway motion cancellation, a gait, or any combination thereof. When the resident is in bed, the determined motion can also include a rollover in bed motion, a falling out of bed motion, etc. Examples of how to detect a resident falling out of bed can be found in, for example, WO 2017/032873, which is hereby incorporated by reference herein in its entirety. In some cases, detecting a resident falling out of bed can include using one or more sensors (e.g., a sensor wearable by the user) to capture a physiological parameter of the user, which can be used to determine motion data of the user associated with falling out of the bed.

In some implementations, a large movement that is unexpected (as determined based on prior analysis of paths for the resident, such that the resident is expected to have a high likelihood of traversing a specific region of the sensing field) that is detected by analyzing, using the control system 110, data generated by one or more of the sensors 250 (e.g., the motion sensor 253, the IRUWB sensor 256, etc.) in combination with a change in amplitude of a breathing signal of the resident (detected from data from one or more of the sensors 250) may be indicative of a fall by the resident. That is, an unexpected movement coupled with an increased breathing signal can indicate a fall. Threshold levels may be determined for both signals, for example, based on previous data for the resident and/or for other residents (e.g., other residents that have one or more characteristics in common with the resident in question). The measured movement and/or respiratory amplitude can then be compared with the respective threshold(s).

In some implementations, the system 100 analyses data generated by one or more of the sensors 250 (e.g., the IRUWB sensor 256) to estimate a floor surface type. For example, the system 100 can determine that the floor surface is carpeted (e.g., low pile carpet, high pile carpet, etc.), includes one or more mats, is a wood surface, is a vinyl surface, is a marble/stone surface, etc. In such implementations, the determined floor surface type can be used by the system 100 to calculate the severity of a fall in the area. For example, a fall on a stone floor surface is likely to be more severe than a fall on a high pile carpet surface.

The machine learning algorithms of the present disclosure can include Bayesian analysis, decision trees, support vector machines (SVM), Hidden Markov Models (HMM), neural networks (such as shallow or deep CNN, CNN and LSTM, RNN, auto encoder, hybrid model, etc.), etc., or any combination thereof. Features of the machine learning algorithms of the present disclosure can include temporal, frequency, time-frequency (such as short time Fourier transform or wavelets), etc., or be learned such as by a deep belief network (DBN).

The system 100 can detect one or more residents simultaneously by utilizing multiple transmit and receive pathways, such as to isolate a movement in more than one plane. Movement of a person or persons in the sensing environment can be separated based on movement speed, direction, and periodicity—such as to reject the potentially confounding movement of fans (such as ceiling, box, pedestal, part of HVAC etc.), swaying blinds or curtains, strong air currents, and the movement of other biometrics with distinct size, heart, breathing, and motion signatures such as cats, dogs, birds, etc., or water droplets such as when a shower or faucet is running.

In some implementations, multiple Doppler movements of a person (e.g., swinging arms, moving legs, possible movement of an aid such as a walking stick) can be processed using a neural network of the system 100, such as a deep neural network, in order to classify the quality of gait as, for example, steady gait, unsteady gait, aided gait, unaided gait, etc., or any combination thereof.

In some implementations, the system 100 analyses data from one or more of the sensors 250 to detect a relative decline in movements for a resident over time and a speed of such decline (e.g., including gait parameters, stride length, cadence, speed and so forth). As such, the system 100 is able to cause one or more actions in response to such a detection (e.g., sending a message to a third party, scheduling therapy for the resident, notifying a member of the resident's care team, etc.).

In some implementations, the system 100 processes multiple 3D spectrograms, and processes moving peaks, detects biometrics in the candidate regions, to form paths. The system 100 can track multiple paths simultaneously and in three dimensions. A deep learning model can employ multiple processing layers to learn approximate path, movement, and biometric representations automatically. In some implementations, the system 100 does not require any specific calibration, and can learn the presence of multiple static reflections, even when multiple moving targets are in the detection field from start-up. For example, spectrogram representations of a demodulated RADAR response (such as from a time of flight analysis) with labeled training data (annotated paths, and simulated falls) may be fed into an RNN in order to train a system. For example, a 3D convolution layer(s) may be used, such as applying sliding cuboidal convolution filters to 3D input, whereby the layer convolves the input by moving the filters along the input vertically, horizontally, and along the depth, computing the dot product of the weights and the input, and then adding a bias term. This extends a 2D layer by including depth. The approach can be used to recognize the complex RF scattering of one or more moving persons, such as when using a UWB sensor, and compute gait parameters, biometrics, sitting, standing, walking, lying, fallen, about to fall, at increased risk of falling, and so forth.

In some implementations, system 100 can track a resident's path using multiple sensors located in different parts of space (e.g. a room, or an apartment or dwelling comprising multiple rooms, etc.), even when there is sensing overlap. The system can manage the handover between the sensors, such that one or more resident biometrics and estimated paths are described. For example, two sensors might be in the bedroom: one covering the majority of the room and a second located near the bed for high fidelity sleep sensing. A third sensor may be located in a bathroom. The system can track the resident across the entire space even if “visible” to only one or a subset of the sensors at any one time.

The system 100 can be implemented in a single dwelling room, multiple rooms, a single building, and/or multiple buildings. Further, the system 100 can be used to track and/or monitor mobile residents, limited mobility residents (e.g., residents in wheel chairs or residents using a walking aid), and/or non-mobile residents (e.g., residents confined within a bed). That is, even when there is limited motion sensed by the system 100, such as for a resident that is confined to a bed, or a wheelchair, the system 100 can still predict falls from the bed or from the wheelchair before the resident exits or attempts to exit the bed or wheelchair. In some such implementations, the system 100 can predict such falls by analyzing data generated by one or more of the sensors 250 and/or one or more other sensors. For example, analysis of blood pressure values (e.g., if the person is at risk of orthostatic hypotension), heart rate parameters (detected bradycardia, tachycardia, atrial fibrillation, atrial flutter, palpitations etc.), and respiration parameters (elevated breathing rate from normal during sleep), can be made to aid in detecting a likelihood of light-headedness of a resident. The reason for awakening from sleep can also be analyzed and/or processed (e.g., was the resident startled, did the resident move direct from deep or REM sleep suddenly to awake, etc.). For example, if the resident has a REM behavior disorder, the resident may be more likely to fall from bed. Other risk factors that can be considered in a fall prediction calculation can include a recent heart attack or stroke for the resident.

In some implementations, such as for bed falls, the system 100 learns the relative position of bed in relation to the sensor. This relative position could be inferred or programmed in a setup phase, or learned over time based on bed entry and exit routines. The system 100 can also learn over time the typical movements patterns for an individual during sleep. Based on understanding at first whether a person is asleep or awake, detected movements during sleep can then be analyzed to determine if the resident is moving close to bed edge and at risk of bed fall. If at risk, an alarm is sent (e.g., to a care provider or to the resident, such as via a red light or voice notification or alert tone) to minimize the resident's risk of falling.

In some implementations, the system 100 can connect, via the control system 110, to an electronic medical/health record for one or more residents and share fall prediction and fall detection data. Additionally, the system 100 can receive data from the electronic medical/health record (e.g. EMR system 1260) for a resident, such as, for example, has the resident fallen before, how recently did this occur, in what setting, the severity, the recovery time, and so forth. The system 100 can predict risk of a fall in advance of a fall (e.g., a day, a week, a month, 3 months etc.), and recommend steps to reduce and/or manage or mitigate this risk. This can include a handover to a clinician workflow, such as recommending low to moderate physical training, balance skills, Timed Up and Go test (TUG), a link to digital virtual coaching, physiotherapy, and so forth.

In an example of certain aspects of the present disclosure, a system can monitor a resident in a care facility for fall risk. A fall risk score can be dynamically determined for the resident, such that at any given time, a caretaker (e.g., nurse, physician, or other) can view the resident's current fall risk score on a dashboard display. The resident may be given medication, in which case the medication may be added to the resident's electronic medical record. In some cases, an indication that the medication was properly taken (e.g., as witnessed by a caretaker or detected by a sensor) may be included. The system can update the fall risk score for the resident dynamically based on the patient's taking of the medication. Additionally, the system estimate the pharmokinetics (e.g., a curve of effect of the medication) for the resident generally (e.g., general pharmokinetics for any given individual or group of individuals) or specifically (e.g., specific pharmokinetics for that particular resident, such as determined through modeling and/or sensor data). The system can dynamically update the fall risk score based on the pharmokinetics, such that as the medication is predicted to wear off, the fall risk score may be adjusted accordingly. In this example, if the resident were to take the medication before falling asleep and then wake up in the middle of the night to use the restroom, the system can provide a particular fall risk score based on the estimated effect the medication would have on the resident at that particular time. In such cases, dynamically updating the fall risk score can also be based on other information, such as the sleep stage of the individual when the resident woke up. Further, continual monitoring of sensor data can allow the system to detect when the resident attempts to exit the bed, detect gait information as the resident attempts to move to the restroom, detect posture information before and during the resident's attempt to move to the restroom, and/or detect other such information. Thus, the system can dynamically update the fall risk score based on such detected information. Additionally, the system can use incoming sensor data (e.g., via detected gait information and the like) to learn and improve its predictions and fall risk score calculations. For example, if the system expects a high fall risk score for the resident based on the medication taken and the time the resident woke to use the restroom, but the system detects gait information suggestive that the resident is easily moving to the restroom and not exhibiting a high risk for falling, the system can learn (e.g., update settings, parameters, models, and the like) to improve future predictions, such as giving less weight to the effect of the medication and/or the waking time. In some cases, the system can also dynamically update the fall risk score based on environmental conditions (e.g., ambient temperature or humidity) and other changes in environment (e.g., use in a first facility versus use in a second, different facility). Thus, in an example, all other things being equal, the fall risk score for a resident in a first facility may be different than the fall risk score for that same resident in a second facility. One or more elements or aspects or steps, or any portion(s) thereof, from one or more of any of claims 1-150 below can be combined with one or more elements or aspects or steps, or any portion(s) thereof, from one or more of any of the other claims 1-150 or combinations thereof, to form one or more additional implementations and/or claims of the present disclosure.

While the present disclosure has been described with reference to one or more particular implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1-44. (canceled)

45. A method for predicting a fall using machine learning, the method comprising:
accumulating data associated with movements of a resident of a facility, the accumulated data including accumulated historical data and current data; and
training a machine learning algorithm with the accumulated historical data such that the machine learning algorithm is configured to:
receive as an input the current data, and
determine as an output a predicted time or a predicted location that the resident will experience a fall.

46. The method of claim 45, wherein the accumulated historical data further includes data associated with movements and fall events for a plurality of other people.

47. The method of claim 45, wherein the accumulated historical data further includes data associated with movements associated with one or more static objects.

48. The method of claim 45, wherein the current data includes data associated with (i) time it takes the resident to go from point A to point B, (ii) a time it takes the resident to get out of bed, (iii) a time it takes the resident to get out of a chair, (iv) a time it takes the resident to get out of a couch, (v) a shortening of a stride of the resident, (vi) a deterioration of a stride of the resident, or (vii) any combination of (i) to (vi).

49. The method of claim 45, wherein the machine learning algorithm is configured to determine as the output the predicted location that the resident will experience the fall.

50. The method of claim 45, wherein the machine learning algorithm is further configured to determine a likelihood that the resident will experience the fall.

51. The method of claim 50, responsive to the determined likelihood satisfying a threshold, the method further comprises causing an operation of one or more electronic devices to be modified, wherein modification of the operation of the one or more electronic devices is selected to decrease the likelihood that the resident will experience the fall.

52. The method of claim 51, wherein the one or more electronic devices include a configurable bed apparatus, the configurable bed apparatus including first and second moveable guard rails configured to aid in preventing the resident from falling out of the configurable bed apparatus, wherein modification of the configurable bed apparatus includes moving the first movable guard rail into a position to prevent the resident from falling out of the configurable bed apparatus and moving the second movable guard rail to avoid entrapping the resident in the configurable bed apparatus.

53. The method of claim 51, wherein the one or more electronic devices include a smart sole in a shoe to configured to adjust a gait of the resident to aid in preventing the resident from falling.

54. The method of claim 51, wherein the one or more electronic devices include at least one selected from the group consisting of:
(i) an illumination device configured to be actuated to aid in reducing a likelihood of the resident falling;
(ii) a speaker configured to provide auditory guidance to aid in preventing the resident from falling; and
(iii) a multi-colored illumination device configured to modify a color or an intensity of electromagnetic radiation.

55-56. (canceled)

57. A method for predicting when a resident of a facility will fall, the method comprising:
generating, via a sensor, data associated with movements of a resident, the data including current data and historical data;
receiving, as an input to a machine learning fall prediction algorithm, the current data; and
determining, as an output of the machine learning fall prediction algorithm, (i) a predicted time in the future within which the resident is predicted to fall and (ii) a percentage likelihood of occurrence for the fall.

58. The method of claim 57, further comprising generating, via the sensor or one or more other sensors, historical data associated with movements and fall events for each of a plurality of other people.

59. The method of claim 57, wherein the current data includes data associated with (i) a time it takes the resident to go from point A to point B, (ii) a time it takes the resident to get out of bed, (iii) a time it takes the resident to get out of a chair, (iv) a time it takes the resident to get out of a couch, (v) a shortening of a stride of the resident, (vi) a deterioration of a stride of the resident, or (vii) any combination of (i) to (vi).

60. The method of claim 57, wherein further comprising determining a predicted location for the fall.

61. The method of claim 57, further comprising, responsive to the determined percentage likelihood of occurrence for the fall exceeding a threshold, causing an operation of one or more electronic devices to be modified, wherein modification of the operation of the one or more electronic devices is selected to decrease the likelihood that the resident will experience the fall.

62. The method of claim 61, wherein the one or more electronic devices include a configurable bed apparatus, the configurable bed apparatus including first and second moveable guard rails configured to aid in preventing the resident from falling out of the configurable bed apparatus, wherein modification of the configurable bed apparatus includes moving the first movable guard rail into a position to prevent the resident from falling out of the configurable bed apparatus and moving the second movable guard rail to avoid entrapping the resident in the configurable bed apparatus.

63. The method of claim 61, wherein the one or more electronic devices include a smart sole in a shoe configured to adjust a gait of the resident to aid in preventing the resident from falling.

64. The method of claim 61, wherein the one or more electronic devices include at least one selected from the group consisting of:
(i) an illumination device configured to be actuated to aid in reducing a likelihood of the resident falling;
(ii) a speaker configured to provide auditory guidance to aid in preventing the resident from falling; and
(iii) a multi-colored illumination device configured to modify a color or an intensity of electromagnetic radiation.

65-66.

67. A method for assessing fall risk, comprising:
receiving sensor data associated with an environment in which a resident is located;
analyzing the sensor data;
generating a fall inference associated with the resident based on the analyzed sensor data, wherein the fall inference is indicative of an occurrence of a fall event or a likelihood that the fall event will occur; and
transmitting a signal in response to generating the fall inference, wherein the signal, when received, generates an alert on a display device, wherein the alert identifies that the resident has fallen or that the resident has a high likelihood of falling.

68. The method of claim 67, wherein the fall inference further comprises a location where the fall is likely to occur.

69. The method of claim 67, wherein the fall inference is indicative that a fall event will occur, and wherein the fall inference further comprises additional information selected from the group consisting of a time window when the fall is likely to occur, a time of day when the fall is likely to occur, and an activity associated with when the fall is likely to occur.

70. The method of claim 67, further comprising calibrating the sensor data based on the analyzed sensor data, wherein calibrating the sensor data comprises receiving sensor data associated with the resident and other individuals in the environment.

71. The method of claim 67, wherein transmitting the signal further comprises actuating an actuatable element of an assistance device associated with the resident, wherein actuation of the actuatable element of the assistance device is configured to affect a gait or position of the resident to reduce a likelihood of falling.

72. The method of claim 67, further comprising receiving physiological data associated with the resident, wherein analyzing the sensor data comprises analyzing the sensor data and the physiological data, and wherein generating the fall inference is based on the analyzed sensor data and the analyzed physiological data.

73-76. (canceled)

77. The method of claim 67,
wherein analyzing the sensor data comprises:
identifying gait information associated with the resident, the gait information including pathway information of the resident; and
generating a gait score based on the identified gait information, the gait score being indicative of an amount of deviation from an expected pathway present in the pathway information, and
wherein generating the fall inference further comprises using the gait score.

78-82. (canceled)

83. The method of claim 67, wherein the fall inference comprises a fall inference score, the method further comprising determining a risk stratification level associated with the fall inference score, wherein the alert on the display device comprises the risk stratification level.

84-90. (canceled)

91. The method of claim 67, further comprising accessing health data associated with the resident, wherein the health data comprises a diagnosis associated with the resident, and wherein generating the fall inference comprises adjusting an interpretation of sensor data based on the diagnosis.

92-102. (canceled)

103. The method of claim 67, wherein the sensor data includes data associated with the environment itself, wherein analyzing the sensor data includes determining environmental information associated with the environment, and wherein generating the fall inference based on the analyzed sensor data is based on the environmental information.

104. The method of claim 103, wherein the data associated with the environment itself includes (i) an ambient temperature of the environment, (ii) a humidity of the environment, (iii) a light level of the environment, or (iv) any combination of (i) to (iii).

105-152. (canceled)

153. The method of claim 67, wherein analyzing the sensor data includes identifying information associated with at least one static object in the environment and determining an expected pathway of the resident within the environment, wherein generating the fall inference is based at least in part on the information associated with the at least one static object and the expected pathway of the resident.
